(19) United States
(12) Patent Application Publication
(10) Pub. No.: US 2022/0361778 A1
(43) Pub. Date: Nov. 17, 2022

(54) Data-Stream Bridging for Sensor Transitions

(71) Applicant: Dexcom, Inc., San Diego, CA (US)

(72) Inventors: Lauren H. Jepson, San Diego, CA (US); Nathaniel D Heintzman, San Diego, CA (US); Joost Van der Linden, San Diego, CA (US); Apurv Ullas Kamath, San Diego, CA (US); Anna C Harley-Trochimczyk, San Diego, CA (US); Vincent P Crabtree, San Diego, CA (US); Benjamin E West, San Diego, CA (US); Mark D Kempkey, San Diego, CA (US); Kyoung-Ho Kim, San Diego, CA (US); Craig Thomas Gadd, San Diego, CA (US); Svetlana Whitley, San Diego, CA (US); Maria NB Wells, San Diego, CA (US); Christopher M Popp, San Diego, CA (US); Andrew M Reinhardt, San Diego, CA (US)

(73) Assignee: Dexcom, Inc., San Diego, CA (US)

(21) Appl. No.: 17/746,048

(22) Filed: May 17, 2022

Related U.S. Application Data
(60) Provisional application No. 63/189,429, filed on May 17, 2021.
(60) Provisional application No. 63/231,502, filed on Aug. 10, 2021.

(51) Int. Cl. A61B 5/145; A61B 5/05; A61B 5/1473; A61B 5/1486; A61B 5/00; A61M 5/172

(52) U.S. Cl. CPC A61B 5/14532; A61B 5/05; A61B 5/1473; A61B 5/14865; A61B 5/7257; A61B 5/7264; A61B 5/7275; A61M 5/1723; A61B 5/7445

(57) ABSTRACT

Data-stream bridging for sensor transitions is described. A first data stream of glucose measurements is received from a first glucose sensor worn by a user. A termination event for the first glucose sensor is detected when production and/or communication of the first glucose measurements via the first data stream ceases. Next, a second data stream of glucose measurements is received from a second glucose sensor worn by the user that replaces the first glucose sensor. During a warmup period for the second glucose sensor, estimated glucose values are output for the user based on both the first data stream of glucose measurements received from the first glucose sensor prior to the termination event and the second data stream of glucose measurements received from the second glucose sensor.

### RELATED APPLICATION

This application claims the benefit of U.S. Provisional Patent Application No. 63/189429, filed May 17, 2021, and titled “Data-Stream Bridging for Sensor Transitions,” and U.S. Provisional Patent Application No. 63/231502, filed Aug. 10, 2021, and titled “Data-Stream Bridging for Sensor Transitions,” the entire disclosures of each of which are hereby incorporated by reference.

### BACKGROUND

Diabetes is a metabolic condition affecting hundreds of millions of people. For these people, monitoring blood glucose levels and regulating those levels to be within an acceptable range is important not only to mitigate long-term issues such as heart disease and vision loss, but also to avoid the effects of hyperglycemia and hypoglycemia. Maintaining blood glucose levels within an acceptable range can be challenging, as this level is almost constantly changing over time and in response to everyday events, such as eating or exercising. Advances in medical technologies have enabled development of various systems for monitoring blood glucose, including continuous glucose monitoring (CGM) systems, which measure and record glucose concentrations in substantially real-time. CGM systems are important tools for users of these systems to ensure that measured glucose values are within the acceptable range.

Many glucose monitoring systems utilize wearable devices which include sensors that can be inserted into the skin of a user to monitor glucose. Oftentimes these sensors are disposable and designed to operate for a predetermined amount of time (e.g., ten days), after which the sensor must be replaced with a new sensor. When a new sensor is inserted into the user's skin, the sensor may require a significant amount of time (e.g., two hours) to “warmup” before the sensor is able to consistently produce accurate measurements. During this time period, i.e., during the warmup period, glucose measurements produced using a newly inserted glucose sensor may not be accurate and/or may not be consistently accurate.

Due to the unreliability of glucose sensors during the warmup period, many conventional glucose monitoring systems simply refrain from displaying any type of glucose data during this period. However, the lack of available glucose data during the transition period between sensors results in glucose alerts and alarms being disabled, which can lead to health risks for users who rely on the glucose data to make important decisions, such as when to administer insulin. Consequently, users may resort to using painful finger sticks during the transition period in order to monitor their glucose levels. Resorting to finger sticks can also be inconvenient or annoying and may limit the activities those users can undertake during the warmup period.

### SUMMARY

To overcome these problems, data-stream bridging for sensor transitions is described. A first data stream of glucose measurements is received from a first glucose sensor worn by a user. The first data stream corresponds to a first time period during which a wearable glucose monitoring device produces glucose measurements and streams those measurements (e.g., communicates them) to a computing device associated with the user. A termination event for the first glucose sensor is detected when production and/or communication of the first glucose measurements via the first data stream ceases. Next, a second data stream of glucose measurements is received from a second glucose sensor worn by the user that replaces the first glucose sensor. During a warmup period for the second glucose sensor, estimated glucose values are output for the user based on both the first data stream of glucose measurements received from the first glucose sensor prior to the termination event and the second data stream of glucose measurements received from the second glucose sensor.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an exemplary implementation that is operable to employ techniques described herein.

FIG. 2 depicts an example of a wearable glucose monitoring device in greater detail.

FIG. 3 depicts an example of a system to generate estimated glucose values for a warmup period of a glucose sensor.

FIG. 4 depicts an example implementation of a user interface displaying plots of a user's glucose over time including estimated glucose values for a warmup period of a glucose sensor.

FIG. 5 depicts an example of a scenario in which a bridging system generates estimated glucose values during a transition period between sensors.

FIG. 6 depicts an example implementation of a user interface displaying plots of a user's glucose over time including during a transition period between glucose sensors.

FIG. 7 depicts an example of a scenario in which a bridging system generates estimated glucose values retrospectively to replace glucose measurements at an end of a first sensor's data stream.

FIG. 8 depicts a procedure in an example implementation in which estimated glucose values are output based on both a first stream of glucose measurements received from a first glucose sensor prior to a termination event and a second data stream of glucose measurements received from a second glucose sensor that replaces the first glucose sensor.

FIG. 9 depicts a procedure in an example implementation in which glucose measurements associated with a first glucose sensor are retrospectively updated based on glucose measurements received from a second glucose sensor that replaces the first glucose sensor.

FIG. 10 depicts a procedure in an example implementation in which a warmup period for a new glucose sensor is ended when glucose measurements received from the new glucose sensor satisfy an accuracy threshold.

FIG. 11 illustrates an example of a system including various components of an example device that can be implemented as any type of computing device as described and/or utilized with reference to FIGS. 1-10 to implement embodiments of the techniques described herein.

### DETAILED DESCRIPTION

### Overview

Data-stream bridging for sensor transitions is described. In accordance with the described techniques, a bridging system is configured to “bridge-the-gap” between sensors, such as when a user replaces a first glucose sensor with a second glucose sensor. Doing so enables the system to output estimated glucose values for the user during a sensor transition period which corresponds to the period of time between when an old sensor expires (e.g., a period of time after the old sensor is no longer used for reporting data for the user and/or a period of time when the old sensor is close (e.g., 1-hour prior, 2-hours prior, 12-hours prior, etc.) to no longer being used for reporting data for the user) and a new sensor is properly warmed up and calibrated. The bridging system may also be utilized to reduce the length of the warmup period for a new sensor. As discussed in greater detail throughout, the warmup period for a sensor may be any amount of time (e.g., substantially no time, 0-1 hours, 1-2 hours, greater than 2 hours, etc.).

In one or more implementations, the bridging system receives a first data stream of glucose measurements from a first glucose sensor worn by a user. The first data stream corresponds to a first time period during which a wearable glucose monitoring device produces glucose measurements and streams those measurements (e.g., communicates them) to a computing device associated with the user. The bridging system detects or determines a termination event for the first glucose sensor which corresponds to a time when production and/or communication of the first glucose measurements via the first data stream ceases and/or is due to cease (e.g., a “grace period” prior to actual sensor expiration).

The first sensor may be disposable and designed to operate for a predetermined amount of time (e.g., ten days), for instance, or until occurrence of some event is detected (e.g., signal quality degrades below a quality threshold, a variance of the glucose measurements exceeds a variance threshold, or a chemical reaction occurs). In some cases, therefore, the bridging system detects the termination event when a timer associated with the first glucose sensor expires indicating that the predetermined amount of time has passed. Alternatively or additionally, the termination event can be detected when the user physically removes the first glucose sensor. For example, the first glucose sensor may be inserted subcutaneously into the user's skin, and the termination event may be detected when the first glucose sensor is removed from the user's skin which results in a loss of connectivity between the first sensor and the computing device.

Regardless of the way in which the termination event is detected or determined, the sensor bridging system is configured to “bridge-the-gap” during a transition period between sensors (e.g., it is capable of providing data while a second sensor intended to replace a first sensor is warming up). This transition period between sensors may be a period of time during which a plurality of sensors (e.g., two of them) are simultaneously implanted within a body of a user and/or a period of time where only one sensor (or no sensor) is implanted within the body of the user. In one example, this transition period may extend from expiration of a first sensor to insertion of a second sensor. In another example, this transition period may extend from a grace period prior to expiration of a first sensor to insertion of a second sensor. In another example, the transition period may extend from expiration of a first sensor to after warmup of a second sensor. In another example, the transition period may extend from a grace period prior to expiration of a first sensor to after warmup of a second sensor. Any desired amount of time for the transition period may be used in accordance with the concepts discussed throughout. For example, the transition period or a portion of the transition period may be a predetermined amount of time. In another example, the transition period or a portion of the transition period may be a patient-specific amount of time that is determined based on one or more data streams from one or more sensors and/or other data inputs.

Notably, the transition period between sensors may include both a first time period that begins when the termination event of the first glucose sensor is detected and ends when the second sensor is initiated (e.g., inserted into the user's body) as well as a second time period during which the second glucose sensor warms up. Notably, during the first time period there are no glucose measurements being received, while during the second time period, i.e., during the warmup period, glucose measurements produced using a newly inserted glucose sensor may not be accurate and/or may not be consistently accurate. Due to the fact that the measurements produced by a glucose sensor during the warmup period may not accurately estimate the person's actual glucose level for a significant portion of that time period, most conventional systems simply refrain from outputting estimated glucose values for the user until the warmup period is finished. However, preventing the output of glucose data during the transition between sensors can lead to frustration for users who closely monitor their glucose levels as well as health risks for some users who are unable to view their glucose data for an extended period of time during the transition between sensors.

In order to solve this problem, the bridging system described herein is configured to generate and output a set of estimated glucose measurements during the transition period between sensors. To do so, the bridging system generates estimated glucose values for the user based on both the first glucose measurements received from the first glucose sensor prior to the termination event and second glucose measurements received from a second glucose sensor that replaces the first glucose sensor. By way of example and not limitation, the bridging system may provide the first glucose measurements and the second glucose measurements—or data representative of those measurements (e.g., feature vectors)—as input to one or more machine learning models. Such machine learning models may be configured to predict a current glucose level of a user based on historical glucose measurements of the user from an earlier time period (e.g., the first glucose measurements) and based on relatively current but potentially unsuitable measurements of the user (e.g., the second glucose measurements). In some cases, the model may also leverage other data streams in order to form more accurate glucose predictions, such as by leveraging temperature data, activity data, food logging data, and so forth. By generating and outputting the estimated glucose measurements, the bridging system may “bridge” at least part of the gap in production of accurate glucose measurements that occurs during the transition period. In this way, the user is able to view estimated glucose values, as well as other glucose-related information (e.g., alerts and visualizations) in an uninterrupted manner during the transition period between sensors.

In one or more implementations, the bridging system is configured to reduce the amount of time that a warmup period for a new glucose sensor lasts. In order to reduce the warmup period for a new glucose sensor, the bridging system determines whether a new data stream of glucose measurements received from a new glucose sensor satisfies an accuracy threshold based at least in part on a previous data stream of glucose measurements received from the previous glucose sensor prior to a termination event for the previous glucose sensor. Then, the warmup period for the new glucose sensor is ended when the accuracy threshold is satisfied. Notably, by using accuracy of the glucose measurements received from the new sensor to determine the warmup time, the warmup time may be shortened in relation to a predetermined amount of time. For example, the warmup time may be shortened from a first predetermined amount of time (e.g., two-hours, 30-minutes, etc.) to an amount of time that is less than the first predetermined amount of time (e.g., less than two-hours, less than 30 minutes, etc., respectively). This enables the user to take action to manage glucose levels sooner than if a predetermined amount of time is used for the warmup period. Moreover, by presenting accurate glucose sooner, potentially dangerous events in relation to the user's health may be avoided. This also enables people relying on wearable glucose monitoring devices to reduce their use of finger sticks thereby eliminating a potentially painful and annoying activity from their lives.

Notably, this approach may also personalize the warmup period for users, as different users may have different warmup accuracy thresholds based on the confidence in the accuracy of the first and second data streams. For example, measuring a high amount of sensor noise in the first data stream may indicate progressive sensor decline. In this case, the system may have less confidence in the first sensor data which may result in a stricter accuracy threshold. Similarly, if a user has a strong reaction in their skin upon insertion of the second glucose sensor, resulting in more noise in the readings, then this could also increase the accuracy threshold.

In the following discussion, an exemplary environment is first described that may employ the techniques described herein. Examples of implementation details and procedures are then described which may be performed in the exemplary environment as well as other environments. Performance of the exemplary procedures is not limited to the exemplary environment and the exemplary environment is not limited to performance of the exemplary procedures.

### Example of an Environment

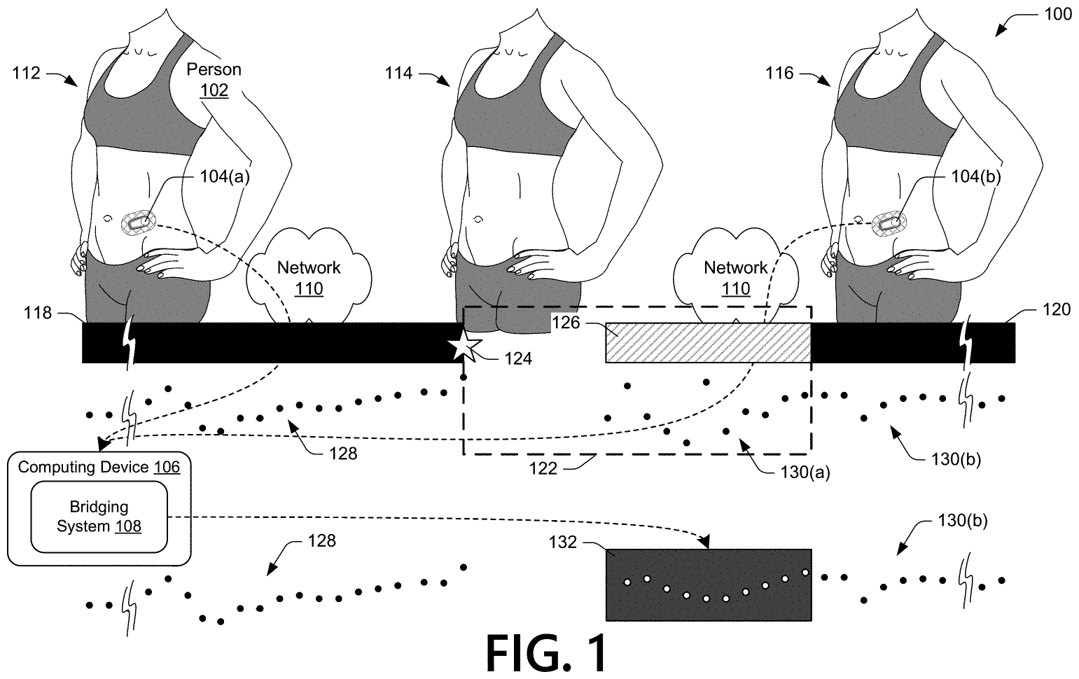

FIG. 1 is an illustration of an environment 100 in an example implementation that is operable to employ data-stream bridging for sensor transitions as described herein. The illustrated environment 100 includes person 102, who is depicted wearing a wearable glucose monitoring device 104, examples of which include wearable glucose monitoring device 104(a) and wearable glucose monitoring device 104(b), having first and second glucose sensors, respectively. The illustrated environment 100 also includes computing device 106, which is depicted having a bridging system 108. The wearable glucose monitoring device 104 and the computing device 106 are communicatively coupled, including via a network 110. Some, all, or none of the components of the illustrated environment 100 in FIG. 1 may be configured to be disposable (e.g., configured for one-time use by a user) or reusable (e.g., configured for use by a user for multiple analyte monitoring sessions).

Alternatively or additionally, the wearable glucose monitoring device 104 and the computing device 106 may be communicatively coupled in other ways, such as using one or more wireless communication protocols or techniques. By way of example, the wearable glucose monitoring device 104 and the computing device 106 may communicate with one another using one or more of Bluetooth (e.g., Bluetooth Low Energy links), near-field communication (NFC), 5G, and so forth.

In accordance with the described techniques, the wearable glucose monitoring device 104 is configured to provide measurements of the person 102's glucose. Although a wearable glucose monitoring device is discussed herein, it is to be appreciated that data-stream bridging may be used for sensor transitions of other devices capable of providing glucose measurements, e.g., non-wearable glucose devices (such as blood glucose meters requiring finger sticks), patches, and so forth. Additionally or alternatively, data-stream bridging may be used in connection with transitions of sensors that produce values different from glucose values, e.g., temperature, other analytes (e.g., lactate, sodium, insulin, etc.), and heart rate, to name just a few. In implementations that involve the wearable glucose monitoring device 104, though, it may be configured with a glucose sensor that detects analytes indicative of the person 102's glucose and enables generation of glucose measurements as discussed above and below.

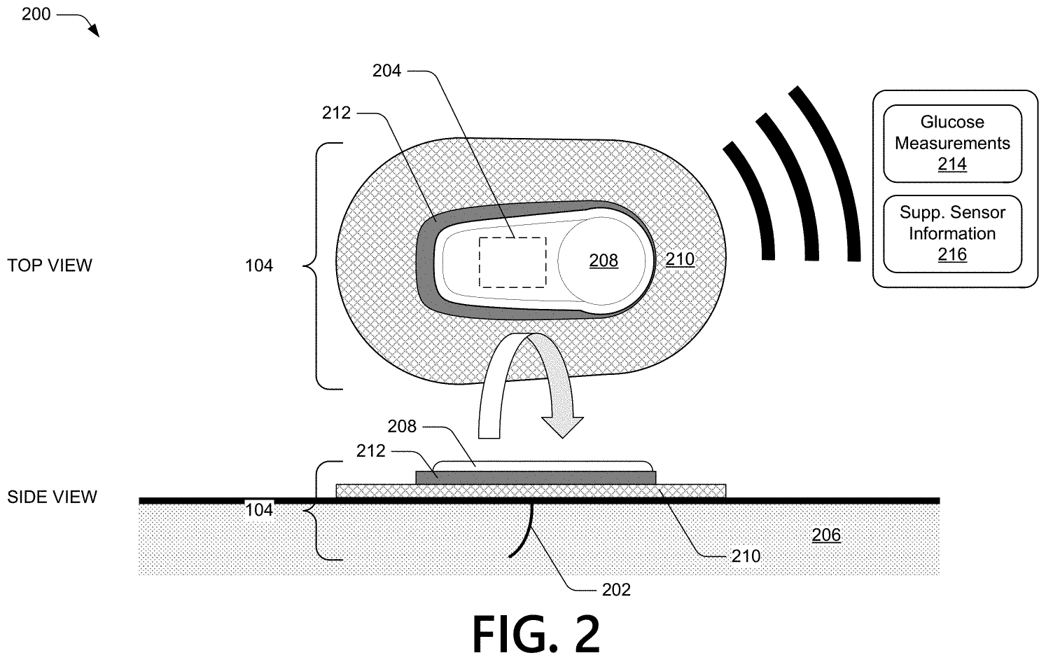

In one or more implementations, the wearable glucose monitoring device 104 is a continuous glucose monitoring (CGM) system. As used herein, the term “continuous” used in connection with glucose monitoring may refer to an ability of a device to produce measurements substantially continuously, such that the device may be configured to produce the glucose measurements at intervals of time (e.g., every hour, every 30 minutes, every 5 minutes, everyone 1 minute, every 30 seconds, etc. and so forth), responsive to establishing a communicative coupling with a different device (e.g., when the computing device 106 establishes a wireless connection with the wearable glucose monitoring device 104 to retrieve one or more of the measurements), and so forth. This functionality along with further aspects of the configuration of the wearable glucose monitoring device 104 are discussed in more detail in relation to FIG. 2.

Additionally, the wearable glucose monitoring device 104 transmits glucose measurements to the computing device 106, such as via a wireless connection. The wearable glucose monitoring device 104 may communicate these measurements in real-time, e.g., as they are produced using a glucose sensor. Alternately or in addition, the wearable glucose monitoring device 104 may communicate the glucose measurements to the computing device 106 at set time intervals. For example, the wearable glucose monitoring device 104 may be configured to communicate the glucose measurements to the computing device 106 every five minutes (as they are being produced). Certainly, an interval at which the glucose measurements are communicated may be different from the examples above without departing from the spirit or scope of the described techniques. The measurements may be communicated by the wearable glucose monitoring device 104 to the computing device 106 according to other bases in accordance with the described techniques, such as based on a request from the computing device 106. Regardless, the computing device 106 may maintain the glucose measurements of the person 102 at least temporarily, e.g., in computer-readable storage media of the computing device 106.

The computing device 106 may be configured in a variety of ways without departing from the spirit or scope of the described techniques. By way of example and not limitation, the computing device 106 may be configured as a mobile device (e.g., a mobile phone, a wearable device, or tablet device), a desktop computer, or a laptop computer, just to name a few form factors. In one or more implementations, the computing device 106 may be configured as a dedicated device associated with a glucose monitoring platform (not shown), e.g., with functionality to obtain glucose measurements from the wearable glucose monitoring device 104, perform various computations in relation to the glucose measurements, display information related to the glucose measurements and the glucose monitoring platform (not shown), communicate the glucose measurements to the glucose monitoring platform, and so forth.

Additionally, the computing device 106 may be representative of more than one device in accordance with the described techniques. In one or more scenarios, for instance, the computing device 106 may correspond to both a wearable device (e.g., a smart watch) and a mobile phone. In such scenarios, both of these devices may be capable of performing at least some of the same operations, such as to receive glucose measurements from the wearable glucose monitoring device 104, communicate them via the network 110 to a glucose monitoring platform, display information related to the glucose measurements, and so forth. Alternatively or in addition, different devices may have different capabilities that other devices do not have or that are limited through computing instructions to specified devices.

In the scenario where the computing device 106 corresponds to a separate smart watch and a mobile phone, for instance, the smart watch may be configured with various sensors and functionality to measure a variety of physiological markers (e.g., heartrate, heartrate variability, breathing, rate of blood flow, and so on) and activities (e.g., steps or other exercise) of the person 102. In this scenario, the mobile phone may not be configured with these sensors and functionality, or it may include a limited amount of that functionality—although in other scenarios a mobile phone may be able to provide the same functionality. Continuing with this particular scenario, the mobile phone may have capabilities that the smart watch does not have, such as a camera to capture images associated with glucose monitoring and an amount of computing resources (e.g., battery and processing speed) that enables the mobile phone to more efficiently carry out computations in relation to glucose measurements. Even in scenarios where a smart watch is capable of carrying out such computations, computing instructions may limit performance of those computations to the mobile phone so as not to burden both devices and to utilize available resources efficiently. To this extent, the computing device 106 may be configured in different ways and represent different numbers of devices than discussed herein without departing from the spirit and scope of the described techniques.

In accordance with the described techniques, the bridging system 108 is configured to “bridge-the-gap” during transitions between sensors, such as when the person 102 replaces a first glucose sensor with a second glucose sensor and/or may include transitions between sensors where the first glucose sensor and the second glucose sensor are simultaneously implanted within the person 102 for an amount of time. In the illustrated environment 100, the person 102 is depicted at three stages, including a first stage 112, a second stage 114, and a third stage 116. Further, a chronological order of the stages may correspond to the first stage 112, followed by the second stage 114, and followed by the third stage 116. In the first stage 112, the person 102 is depicted wearing the wearable glucose monitoring device 104(a) which includes a first glucose sensor. In the second stage 114, the person 102 is depicted not wearing a glucose monitoring device. This may correspond to a period of time after the person 102 removes the wearable glucose monitoring device 104(a), but before the person 102 applies the wearable glucose monitoring device 104(b), e.g., when the person 102 switches from using the first sensor of the wearable glucose monitoring device 104(a) to using a second sensor of the wearable glucose monitoring device 104(b). In the third stage 116, the person 102 is depicted wearing the wearable glucose monitoring device 104(b) which, as noted above, includes the second glucose sensor. Additional and/or different stages may be present in alternative embodiments (e.g., a stage whereby the person 102 is simultaneously wearing the wearable glucose monitoring device 104(a) and the wearable glucose monitoring device 104(b)). In any embodiment, the bridging system 108 may be used to help “bridge-the-gap” between a first data stream associated with the wearable glucose monitoring device 104(a) and a second data stream associated with the wearable glucose monitoring device 104(b) being accurate or “warmed up” for the person 102.

In one or more implementations, the wearable glucose monitoring device 104(a) and the wearable glucose monitoring device 104(b) may share one or more components, e.g., a common transmitter, processor, or computer-readable storage, to name just a few. In such implementations, those shared components may be reusable, such that the reusable components can be coupled with one or more disposable components, e.g., glucose sensors, which are disposed of after a period of time. The disposable components may be designed to operate for a predetermined amount of time (e.g., ten days, fifteen days, etc.), for instance, or until occurrence of some event is detected (e.g., signal quality degrades below a quality threshold, a variance of the glucose measurements exceeds a variance threshold, or a chemical reaction occurs). In this way, the wearable glucose monitoring device 104(b) may use a same transmitter to transmit glucose measurements to the computing device 106 as the wearable glucose monitoring device 104(a).

Alternatively, the wearable glucose monitoring device 104(a) and the wearable glucose monitoring device 104(b) may not share any components—they may be completely separate devices. In such implementations, the wearable glucose monitoring device 104(a) and the wearable glucose monitoring device 104(b) may have a same design and be configured in a same manner, but they may be manufactured as separate devices such that they correspond to a first physical device and a separate, second physical device. For instance, the wearable glucose monitoring device 104(a) may include a first glucose sensor and a first transmitter, and the wearable glucose monitoring device 104(b) may include a second glucose sensor and a second transmitter, which are separate from the first glucose sensor and the first transmitter but have a same configuration as the first glucose sensor and transmitter. Alternatively or additionally, the wearable glucose monitoring device 104(a) and the wearable glucose monitoring device 104(b) may be separate devices configured differently, e.g., different model numbers, having different types of components from one another. In such scenarios, though, separate devices having different designs and configurations nevertheless may be configured to communicate glucose measurements to the computing device 106.

The illustrated environment 100 also depicts first data stream 118, second data stream 120, transition period 122, termination event 124, and warmup period 126. In accordance with the described techniques, the first data stream 118 includes first glucose measurements 128, which are produced by the wearable glucose monitoring device 104(a) using its respective glucose sensor (e.g., the first glucose sensor) and communicated as part of the first data stream 118 to the computing device 106. In a similar manner, the second data stream 120 includes second glucose measurements 130(a), 130(b), which are produced by the wearable glucose monitoring device 104(b) using its respective glucose sensor (e.g., the second glucose sensor) and communicated as part of the first data stream 118 to the computing device 106. Here, the second glucose measurements 130(a) correspond to a portion of the measurements in the second data stream 120 that are produced by the wearable glucose monitoring device 104(b) during the warmup period 126. In contrast, the second glucose measurements 130(b) correspond to a portion of the measurements in the second data stream 120 that are produced by the wearable glucose monitoring device 104(b) after the warmup period 126, e.g., a subsequent portion of the second data stream 120's glucose measurements.

In environment 100, the first data stream 118 and the second data stream 120 are illustrated as blocks to visually convey a time period to which those streams correspond. By way of example, the first data stream 118 corresponds to a first time period during which the wearable glucose monitoring device 104(a) produces the first glucose measurements 128 and streams those measurements (e.g., communicates them) to the computing device 106. The termination event 124 corresponds to a time (e.g., a point in time) when production and/or communication of the first glucose measurements 128 via the first data stream 118 ceases. The various events that may cause or be detected as the termination event 124 are discussed in more detail below.

Further, the illustrated environment 100 includes no “block” between the termination event 124, terminating the first data stream 118, and the portion of the second data stream 120 corresponding to the warmup period 126. This indicates that in one or more implementations, no glucose measurements are produced and communicated by a glucose monitoring device to the computing device 106 over a period of time between the termination event 124 and the warmup period 126 of the wearable glucose monitoring device 104(b). Indeed, this comports with the depiction of the person 102 at the second stage 114 in which the person 102 is depicted not wearing a glucose monitoring device, e.g., after the person 102 has removed the wearable glucose monitoring device 104(a) and before the person 102 has applied the wearable glucose monitoring device 104(b). In some implementations, however, there may be no “break” in production and streaming of glucose measurements—although the glucose measurements may originate from different sources—such as when the wearable glucose monitoring device 104(b) is applied while the person 102 is still wearing the wearable glucose monitoring device 104(a). In this case, the bridging system 108 may receive sensor measurements from both the wearable glucose monitoring device 104(a) and the wearable glucose monitoring device 104(b) at the same time. In other words, the cool down period for the first glucose sensor may overlap the warm-up period for the second glucose sensor. The estimated glucose values may be determined, in this scenario, based on real-time glucose measurements received from two different sensors being worn at the same time.

The second data stream 120 corresponds to a second time period that is subsequent to the first time period. This second time period also corresponds to when the wearable glucose monitoring device 104(b) produces the second glucose measurements 130(a), 130(b) and streams those measurements (e.g., communicates them) to the computing device 106.

The first glucose measurements 128 and the second glucose measurements 130(a), 130(b) are illustrated separately from the first and second data streams 118, 120 to visually convey how measurements of the person 102's glucose may vary over time—even though the first glucose measurements 128 and the second glucose measurements 130(a), 130(b) are included in the first and second data streams 118, 120, respectively. By illustrating the first glucose measurements 128 and the second glucose measurements 130(a), 130(b) separately and as dots, the illustrated environment 100 indicates how measurements of the person 102's glucose can vary over time. The illustrated “dots” may also more closely represent how the computing device 106 outputs (e.g., displays) the person 102's glucose, e.g., as glucose traces.

Notably, the second glucose measurements 130(a) that correspond to the warmup period 126 of the wearable glucose monitoring device 104(b) (e.g., a warmup period of the second glucose sensor) have been illustrated with more variability than the first glucose measurements 128 and the second glucose measurements 130(b) that are produced and communicated after the warmup period 126. This represents that in one or more scenarios, glucose sensors may require an amount of time (e.g., two hours, 30 minutes, etc.) before they are able to be used to consistently produce accurate measurements. During this time period, i.e., during the warmup period 126, glucose measurements produced using a newly inserted glucose sensor may not be accurate and/or may not be consistently accurate.

This may occur because some glucose sensors are designed so that responsive to insertion into a body (e.g., of the person 102), one or more chemical reactions occur. The one or more chemical reactions may enable the sensors to detect analytes in the body indicative of glucose. One example of such a chemical reaction is a substance that coats the glucose sensor dissolving when inserted into the person 102's body over the warmup period 126. Alternatively or additionally, glucose sensors may need to fill with fluid from the person 102's body during the warmup period 126. Indeed, glucose sensors may not be able to produce or may not be relied on to produce accurate and/or consistently accurate measurements of the person 102's glucose during the warmup period for different reasons without departing from the spirit or scope of the described techniques. Regardless, the measurements produced by the wearable glucose monitoring device 104(b) during the warmup period 126 may not accurately estimate the person 102's actual glucose level for a significant portion of that time period, if at all.

In general, the bridging system 108 is configured to generate estimated glucose values for periods of time during which glucose monitoring devices are susceptible to producing unsuitable glucose measurements, e.g., measurements that fail to accurately estimate a person's actual glucose level. In one or more implementations, for example, the bridging system 108 is configured to generate and output a set of estimated glucose values 132 during the warmup period 126 for the glucose sensor of the wearable glucose monitoring device 104(b) (the second glucose sensor discussed above). The bridging system 108 may generate the estimated glucose values 132 for the person 102 based on the first glucose measurements 128 of the first data stream 118 and based on the second glucose measurements 130(a) of the second data stream 120 that correspond to the warmup period 126.

By way of example and not limitation, the bridging system 108 may provide the first glucose measurements 128 and the second glucose measurements 130(a)—or data representative of those measurements (e.g., feature vectors)—as input to one or more machine learning models. Such machine learning models may be configured to predict a current glucose level of a user based on historical glucose measurements of the user from an earlier time period (e.g., the first glucose measurements 128) and based on relatively current but potentially unsuitable measurements of the user (e.g., the second glucose measurements 130(a)).

By generating the estimated glucose values 132, the bridging system 108 may “bridge” at least part of the gap in production of accurate glucose measurements that occurs during the transition period 122. In one or more implementations, the bridging system 108 may also be configured to generate estimated glucose values for the person 102 during the second stage 114, e.g., while the person is not wearing any glucose monitoring device and which corresponds to the time period between the termination event 124 and a start of the warmup period 126. The bridging system 108 may be configured to generate estimated glucose values for the person 102 during this time period based only on the first glucose measurements 128, such as by using one or more machine learning models configured to predict a person's glucose based on historical glucose measurements. To this end, the bridging system 108 may generate estimated glucose values for the person 102 while the person 102 is not wearing a glucose monitoring device (e.g., during the second stage 114) based on the first glucose measurements 128. In contrast, the bridging system 108 may generate the estimated glucose values 132 for the person 102 during the warmup period 126 based on both the first glucose measurements 128 and the second glucose measurements 130(a) produced during the warmup period 126. Additionally or alternatively, the bridging system 108 may leverage other data streams in order to accurately generate the estimated glucose values 132, such as by leveraging temperature data, activity data, food logging data, and so forth.

In one or more implementations, the computing device 106 (e.g., an application associated with the computing device 106) may effectively replace the second glucose measurements 130(a) with the estimated glucose values 132, e.g., for decision support and display. By using the first glucose measurements 128 along with the second glucose measurements 130(a), the bridging system 108 generates a more accurate trace of the person 102's actual glucose (e.g., the estimated glucose values 132) than is represented by the second glucose measurements 130(a). With a more accurate trace, the person 102 or a health care provider of the person 102 may be able to rely on the estimated glucose values 132 during the warmup period 126 to guide treatment decisions, such as whether and what to eat, whether to administer insulin, or whether to contact a health care provider to mitigate a serious health condition, to name just a few. In the context of measuring glucose, e.g., continuously by a continuous glucose monitoring system, and obtaining data describing such measurements, consider the following discussion of FIG. 2.

FIG. 2 depicts an example 200 of an implementation of a wearable glucose monitoring device in greater detail. In particular, the illustrated example 200 includes a top view and a corresponding side view of the wearable glucose monitoring device 104. It is to be appreciated that the wearable glucose monitoring device 104 may vary in implementation from the following discussion in various ways without departing from the spirit or scope of the described techniques. As noted above, for instance, data-stream bridging for sensor transitions may be used in connection with other types of devices for glucose monitoring, such as non-wearable devices (e.g., blood glucose meters requiring finger sticks), patches, and so forth.

In this example 200, the wearable glucose monitoring device 104 is illustrated to include a glucose sensor 202 and a sensor module 204. Here, the glucose sensor 202 is depicted in the side view having been inserted subcutaneously into skin 206, e.g., of the person 102. The sensor module 204 is depicted in the top view as a dashed rectangle. The wearable glucose monitoring device 104 also includes a transmitter 208 in the illustrated example 200. Use of the dashed rectangle for the sensor module 204 indicates that it may be housed or otherwise implemented within a housing of the transmitter 208. In this example 200, the wearable glucose monitoring device 104 further includes adhesive pad 210 and attachment mechanism 212.

In operation, the glucose sensor 202, the adhesive pad 210, and the attachment mechanism 212 may be assembled to form an application assembly, where the application assembly is configured to be applied to the skin 206 so that the glucose sensor 202 is subcutaneously inserted as depicted. In such scenarios, the transmitter 208 may be attached to the assembly after application to the skin 206 via the attachment mechanism 212. Alternatively, the transmitter 208 may be incorporated as part of the application assembly, such that the glucose sensor 202, the adhesive pad 210, the attachment mechanism 212, and the transmitter 208 (with the sensor module 204) can all be applied at once to the skin 206. In one or more implementations, this application assembly is applied to the skin 206 using a separate sensor applicator (not shown). Unlike the finger sticks required by conventional blood glucose meters, the user-initiated application of the wearable glucose monitoring device 104 is nearly painless and does not require the withdrawal of blood. Moreover, the automatic sensor applicator generally enables the person 102 to embed the glucose sensor 202 subcutaneously into the skin 206 without the assistance of a clinician or healthcare provider.

The application assembly may also be removed by peeling the adhesive pad 210 from the skin 206. It is to be appreciated that the wearable glucose monitoring device 104 and its various components as illustrated are simply one example form factor, and the wearable glucose monitoring device 104 and its components may have different form factors without departing from the spirit or scope of the described techniques.

In operation, the glucose sensor 202 is communicatively coupled to the sensor module 204 via at least one communication channel which can be a wireless connection or a wired connection. Communications from the glucose sensor 202 to the sensor module 204 or from the sensor module 204 to the glucose sensor 202 can be implemented actively or passively and these communications can be continuous (e.g., analog) or discrete (e.g., digital).

The glucose sensor 202 may be a device, a molecule, and/or a chemical which changes or causes a change in response to an event which is at least partially independent of the glucose sensor 202. The sensor module 204 is implemented to receive indications of changes to the glucose sensor 202 or caused by the glucose sensor 202. For example, the glucose sensor 202 can include glucose oxidase which reacts with glucose and oxygen to form hydrogen peroxide that is electrochemically detectable by the sensor module 204 which may include an electrode. In this example, the glucose sensor 202 may be configured as or include a glucose sensor configured to detect analytes in blood or interstitial fluid that are indicative of glucose level using one or more measurement techniques. In one or more implementations, the glucose sensor 202 may also be configured to detect analytes in the blood or the interstitial fluid that are indicative of other markers, such as lactate levels, which may improve accuracy in identifying or predicting glucose-based events. Additionally or alternately, the wearable glucose monitoring device 104 may include additional sensors to the glucose sensor 202 to detect those analytes indicative of the other markers.

In another example, the glucose sensor 202 (or an additional sensor of the wearable glucose monitoring device 104—not shown) can include a first and second electrical conductor and the sensor module 204 can electrically detect changes in electric potential across the first and second electrical conductor of the glucose sensor 202. In this example, the sensor module 204 and the glucose sensor 202 are configured as a thermocouple such that the changes in electric potential correspond to temperature changes. In some examples, the sensor module 204 and the glucose sensor 202 are configured to detect a single analyte, e.g., glucose. In other examples, the sensor module 204 and the glucose sensor 202 are configured to detect multiple analytes, e.g., sodium, potassium, carbon dioxide, and glucose. Alternately or additionally, the wearable glucose monitoring device 104 includes multiple sensors to detect not only one or more analytes (e.g., sodium, potassium, carbon dioxide, glucose, and insulin) but also one or more environmental conditions (e.g., temperature). Thus, the sensor module 204 and the glucose sensor 202 (as well as any additional sensors) may detect the presence of one or more analytes, the absence of one or more analytes, and/or changes in one or more environmental conditions.

In one or more implementations, the sensor module 204 may include a processor and memory (not shown). The sensor module 204, by leveraging the processor, may generate glucose measurements 214 based on the communications with the glucose sensor 202 that are indicative of the above-discussed changes. The glucose measurements 214 may correspond to glucose measurements of any of the data streams discussed in relation to FIG. 1, e.g., at least a portion of the first glucose measurements 128 of the first data stream 118 or at least a portion of the second glucose measurements 130(a), 130(b) of the second data stream 120. Certainly, the glucose measurements 214 may correspond to other data streams without departing from the spirit or scope the techniques. Regardless, based on the above-noted communications from the glucose sensor 202, the sensor module 204 is further configured to generate communicable packages of data that include at least one glucose measurement 214. In one or more implementations, the sensor module 204 may configure those packages to include additional data, including, by way of example and not limitation, supplemental sensor information 216. Such supplemental sensor information may include a sensor identifier, a sensor status, temperatures that correspond to the glucose measurements 214, measurements of other analytes that correspond to the glucose measurements 214, and so forth. It is to be appreciated that supplemental sensor information 216 may include a variety of data that supplements at least one glucose measurement 214 without departing from the spirit or scope of the described techniques.

In implementations where the wearable glucose monitoring device 104 is configured for wireless transmission, the transmitter 208 may transmit the glucose measurements 214 and/or the supplemental sensor information 216 wirelessly as a stream of data to a computing device. Alternately or additionally, the sensor module 204 may buffer the glucose measurements 214 and/or the supplemental sensor information 216 (e.g., in memory of the sensor module 204 and/or other physical computer-readable storage media of the wearable glucose monitoring device 104) and cause the transmitter 208 to transmit the buffered glucose measurements 214 and/or buffered supplemental sensor information 216 later at various intervals, e.g., time intervals (every second, every thirty seconds, every minute, every five minutes, every hour, and so on), storage intervals (when the buffered glucose measurements 214 and/or supplemental sensor information 216 reach a threshold amount of data or a number of measurements), and so forth.

Having considered an example of an environment and an example of a wearable glucose monitoring device, consider now a discussion of some examples of details of the techniques for data-stream bridging for sensor transitions in accordance with one or more implementations.

### Data-Stream Bridging for Sensor Transitions

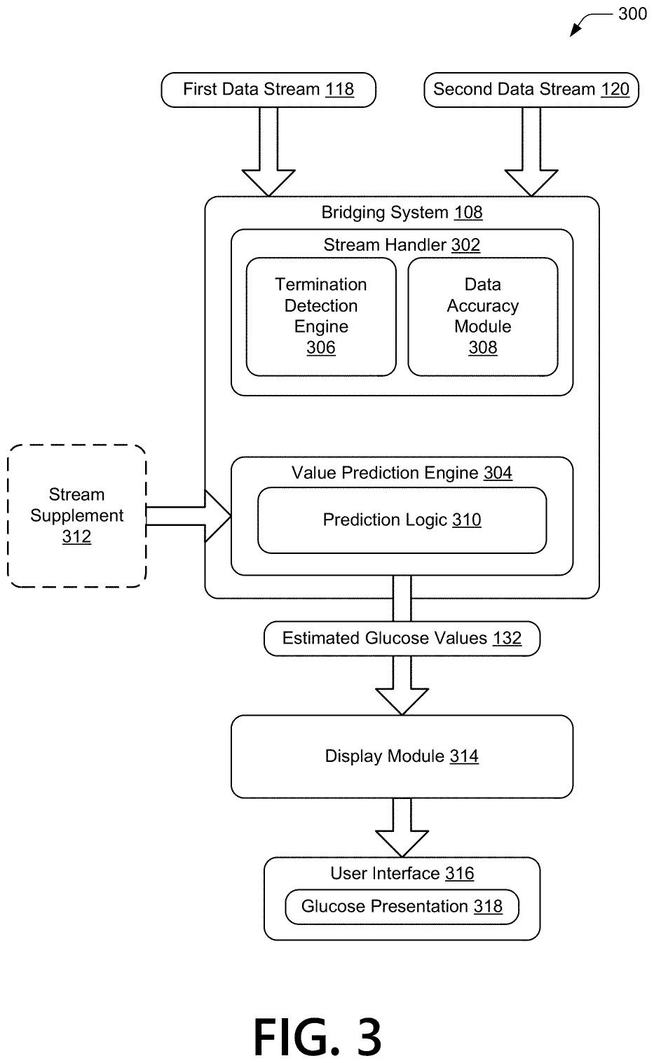

FIG. 3 depicts an example 300 of a system to generate estimated glucose values for a warmup period of a glucose sensor. The illustrated example 300 includes a bridging system 108.

In this example 300, the bridging system 108 is depicted receiving the first data stream 118 and the second data stream 120 as input. The second data stream 120 may be received subsequent to the first data stream 118 and/or may be received simultaneously. The bridging system 108 is also depicted outputting the estimated glucose values 132. As illustrated, the bridging system 108 includes stream handler 302 and value prediction engine 304. The stream handler 302 is depicted including termination detection engine 306 and data accuracy module 308. The value prediction engine 304 is depicted including prediction logic 310. These components are configured to perform various aspects that enable the bridging system 108 to “bridge the gap” for transitions between sensors as discussed above and below. Although the bridging system 108 is depicted with these various components, it is to be appreciated that in implementations the bridging system 108 may include fewer, more, and/or different components without departing from the spirit or scope of the described techniques.

In one or more implementations, the termination detection engine 306 is configured to detect termination events for glucose sensors. In the context of FIG. 1, for example, the termination detection engine 306 is configured to detect the termination event 124. As mentioned above, the termination event 124 may correspond to a time when production and/or communication of the first glucose measurements 128 by the wearable glucose monitoring device 104(a) as part of the first data stream 118 ceases or is due to cease (e.g., a grace period prior to cessation). The termination detection engine 306 may be configured to detect different types of termination events in accordance with the described techniques.

By way of example, glucose sensors may be configured for use over a limited, predetermined amount of time, e.g., 7 days, 10 days, or 30 days, to name just a few. In such implementations, each glucose sensor may be associated with a timer, e.g., that begins when the glucose sensor is inserted into a user's skin or is otherwise deployed to measure the user's glucose. The termination detection engine 306 may maintain the timer and/or detect when the timer expires. In implementations where the timer counts down (decays), for instance, the termination detection engine 306 may detect when the timer reaches zero or falls to some other threshold value indicating an end of life of the respective sensor. In contrast, in implementations where the timer counts up (increases), the termination detection engine 306 may detect when the timer reaches a threshold value (e.g., 10 days) indicating an end of life of the respective sensor.

Alternatively or additionally, the termination detection engine 306 may detect termination events that correspond to changes in conditions that are different from identifying an expiring timer value representing a “lifespan” of a glucose sensor. By way of example, the termination detection engine 306 may detect termination events based on loss of connectivity (e.g., communicative or physical) with a glucose sensor. For instance, the termination detection engine 306 may detect that a termination event has occurred when a glucose measurement is not received from a glucose sensor after a threshold amount of time, when a signal (e.g., a “heartbeat”) is not received from the glucose sensor, when a signal is received indicating an end of connectivity with the glucose sensor, when an end-of-life chemical reaction is detected for a glucose sensor, or when a chemical reaction is detected for a new sensor, to name just a few.

As noted above, glucose sensors of the wearable glucose monitoring devices 104 may be inserted subcutaneously into the person 102's skin. In one or more implementations, the termination detection engine 306 may be configured to detect termination events when those glucose sensors are removed from the skin of the user, e.g., withdrawn from the person 102's skin. In accordance with the described techniques, the termination detection engine 306 is configured to detect the termination event 124, which indicates when production and/or communication of the first data stream 118's glucose measurements ceases. Alternatively or additionally, the termination event may be initiated by user interaction with a user interface (e.g., user interface 316). For example, the user may be able to select a control to end the session with the first glucose sensor and start a new session with the second glucose sensor. It is to be appreciated that the termination detection engine 306 may detect different types of termination events without departing from the spirit or scope of the described techniques.

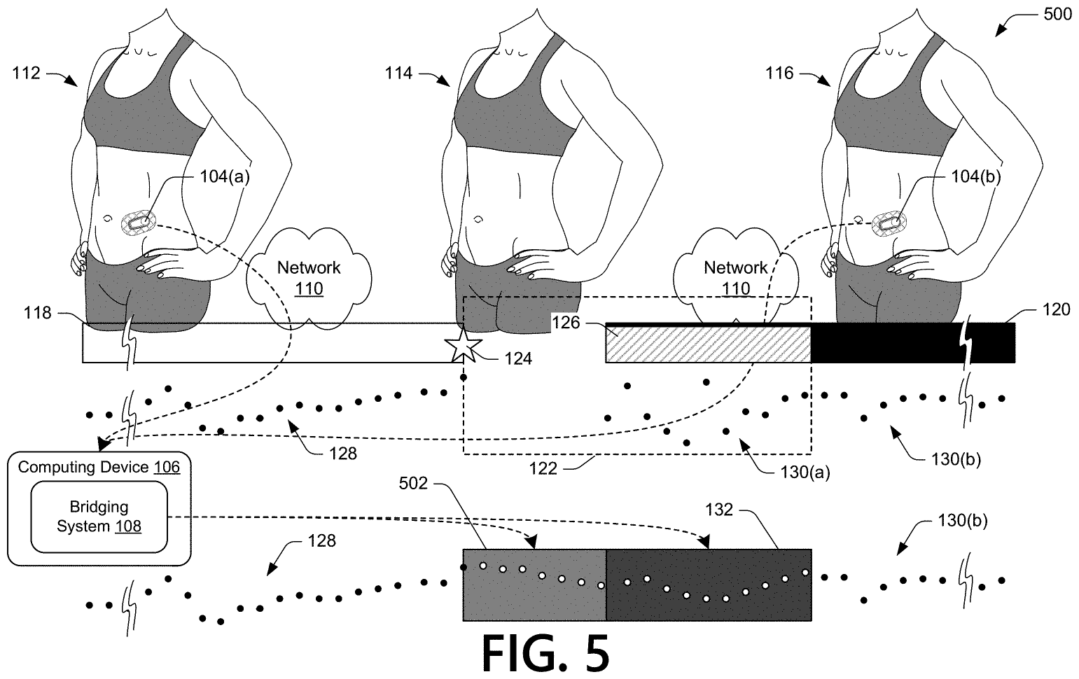

In general, the value prediction engine 304 is configured to use actual glucose measurements produced using glucose sensors to generate the estimated glucose values 132. For example, the value prediction engine 304 may use measurements from one or more of the first data stream 118 or the second data stream 120 to generate the estimated glucose values 132. Additionally, the value prediction engine 304 may generate the estimated glucose values 132 to replace one or more glucose measurements of the first data stream 118 or the second data stream 120. In the context of FIG. 1, for example, the value prediction engine 304 generates the estimated glucose values 132 to replace glucose measurements of the second data stream 120, specifically, to replace the second glucose measurements 130(a) that correspond to the warmup period 126 of the wearable glucose monitoring device 104(b)'s glucose sensor. The value prediction engine 304 may also be used to generate estimated glucose values for other periods of time, as discussed in relation to FIGS. 5-7. These time periods may include, for example, a period of time when a user is not wearing any sensor and retrospectively for a period of time at an end of a sensor's use (or connectivity), e.g., end-of-life of a sensor. Additionally or alternatively, the value prediction engine 304 may leverage other data streams (not shown) in order to accurately generate estimated glucose values, such as by leveraging temperature data, activity data, food logging data, and so forth.

When generating the estimated glucose values 132 for the warmup period 126 of a glucose sensor, the value prediction engine 304 may generate those values based on the first glucose measurements 128 and also based on the second glucose measurements 130(a) that correspond to the warmup period 126. To generate the estimated glucose values 132 from the first glucose measurements 128 and from the second glucose measurements 130(a), the value prediction engine 304 uses the prediction logic 310. In operation, the prediction logic 310 may be configured as executable code (e.g., a “binary”) capable of receiving those glucose measurements as input, processing them according to an encoded algorithm, formula, set of rules, and/or models, and determining and outputting the estimated glucose values 132. The prediction logic 310 may be implemented based on a variety of algorithms, formulas, rules, and/or models without departing from the spirit or scope of the described techniques.

In one or more implementations, for example, the prediction logic 310 may determine the estimated glucose values 132 using one or more weighting techniques, such as weighted averages. For instance, the prediction logic 310 may associate relatively smaller weights with the second glucose measurements 130(a) toward a beginning of the warmup period 126 and may associate relatively larger weights with the second glucose measurements 130(a) closer to an end of the warmup period 126. In one or more implementations, the prediction logic 310 may associate progressively larger weights with the second glucose measurements 130(a) over time. As an example, at a beginning of the warmup period 126, prediction logic 310 may assign a weight of 0.9 to the first glucose measurements 128 and a weight of 0.1 to the second glucose measurements 130(a), and then compute the estimated glucose values 132 as the sum of the weighted first and second glucose measurements. In contrast, towards the end of the warmup period 126, prediction logic 310 may assign a weight of 0.1 to the first glucose measurements 128 and a weight of 0.9 to the second glucose measurements 130(a), and then compute the estimated glucose values 132 as the sum of the weighted first and second glucose measurements.

As part of determining the estimated glucose values 132, the prediction logic 310 may also associate weights with first glucose measurements 128 or with predictions derived from those measurements. For instance, the prediction logic 310 may include one or more machine learning models, trained on historical data of a population of users, to predict a person's glucose for a period of time in the future (e.g., an upcoming 30 minutes, 2 hours, or entire day). Such machine learning models may predict the person 102's glucose responsive to receiving one or more sequences (e.g., traces) of glucose measurements as input (e.g., a last 30 minutes, a last 6 hours, or a last day, of the person 102's glucose measurements). In such implementations, the prediction logic 310 may determine the estimated glucose values 132 based on the glucose values predicted for the warmup period 126 (e.g., predicted from the first glucose measurements 128) and also based on the second glucose measurements 130(a) that correspond to the warmup period 126.

As the prediction logic 310 associates progressively larger weights with the second glucose measurements 130(a) over time, for instance, the prediction logic 310 may also associate progressively smaller weights with the glucose values predicted for the warmup period 126. In this way, the predicted glucose values may more heavily influence the estimated glucose values 132 toward a beginning of the warmup period 126 and the second glucose measurements 130(a) may more heavily influence the estimated glucose values 132 toward an end of the warmup period 126. The weights associated with the predicted glucose values may be said to “decay” while the weights associated with the second glucose measurements 130(a) may be said to “grow” over time. In one or more implementations, the prediction logic 310 may determine the estimated glucose values 132 by computing a weighted average of the glucose values predicted and the second glucose measurements 130(a).

The prediction logic 310 may also determine the estimated glucose values 132 based on stream supplement 312. The stream supplement 312 is illustrated with dashed lines to indicate that its use is optional, e.g., in one or more implementations it may not be used to determine the estimated glucose values 132. Nevertheless, in implementations where the stream supplement 312 is used to determine the estimated glucose values 132, the stream supplement 312 may include data that describes one or more aspects of the person 102, one or more sensors used to obtain measurements, the first or second data streams 118, 120 via which the measurements are communicated to the computing device 106, and/or other factors that may affect an accuracy or reliability of the measurements produced by those sensors and/or received by the computing device 106.

By way of example, aspects of the person 102 that the stream supplement 312 may describe include a temperature of the person 102, foods the person 102 has eaten (e.g., how much, when, types of foods, and/or macros), exercise performed by the person 102 (e.g., how long, intensity, type, and/or performance metrics), sleep of the person 102 (e.g., duration, quality, and/or when), analytes measured for the person 102 in addition to glucose (e.g., insulin, lactate, sodium, potassium, and/or carbon dioxide), physiological markers of the person (e.g., heart rate, heart rate variability (HRV), blood pressure, and/or blood oxygen level), stress of the person 102 (e.g., identification of stressful events), demographics of the person (e.g., age, gender, location, and genetic markers), and identified or diagnosed health conditions of the person (e.g., Type 1 diabetes, Type 2 diabetes, pregnancy, injury, and/or surgeries), to name just a few.

Example aspects of the one or more sensors that the stream supplement 312 may describe include a “state” of a sensor, where the “state” of a sensor refers to a value that the sensor is designed to determine and associate with one or more of the measurements. Such a state indicates whether the sensor is operating “normally” or is operating outside of normal conditions when a respective measurement is produced. Other example aspects of sensors that the stream supplement 312 may describe include, for instance, a manufacturing lot of a sensor, a unique identifier of the sensor, a model number of the sensor, factory calibration information of the sensor, remaining time for which the sensor is to be used to produce measurements, a relative accuracy of the sensor, a relative variability of the sensor, information regarding errors detected in relation to the sensor, a measure of quality of information produced by the sensor, and/or information regarding interfaces of the sensor with other components, to name just a few.

Example aspects of the data streams that the stream supplement 312 may describe include connectivity issues (e.g., loss of connectivity) between the wearable glucose monitoring device 104(a) and the computing device 106, connectivity issues between the wearable glucose monitoring device 104(b) and the computing device 106, corruption detected in relation to one or more portions of the data streams, quality metrics associated with one or more portions of the data stream (e.g., describing a quality of corresponding packets and/or communication of those packets), and delays of one or more portions of the data streams, to name just a few. Indeed, these are just examples of the aspects that may be described by the stream supplement 312, and it may describe a variety of other aspects that affect an accuracy of the measurements produced by the sensors and/or received by the computing device 106 without departing from the spirit or scope of the described techniques.

As noted above, the prediction logic 310 may be implemented based on a variety of algorithms, formulas, rules, and/or models. In implementations where the prediction logic 310 is implemented based on or otherwise includes one or more machine learning models, for instance, at least one of those models may be “general” to a population. Such a model may be trained using training data that describes users of a user population and is trained in order to generate predictions of glucose for various users of the user population. Broadly speaking, a model that is “general” to a population has not been further trained for a specific end user when it is used in operation to generate predictions for the specific end user, e.g., through further training using transfer learning and/or training data of the specific end user. Alternatively or additionally, at least one of the models used by the prediction logic 310 may be specific to an end user. Such a model may be trained using historical data describing the specific end user for which it is trained to generate predictions of glucose. In one or more implementations, such “user specific” models initially may be “general” to a population and then further trained using transfer learning and/or training data describing the specific user.

In one or more implementations, a machine learning model of the prediction logic 310 may receive as input data from the first data stream 118 (e.g., one or more of the first glucose measurements 128), data from the second data stream 120 (e.g., one or more of the second glucose measurements 130(a)), and stream supplement 312, which describes various aspects of the person 102 and/or aspects of the context in which the prediction logic 310 generates the estimated glucose values 132. This machine learning model may then determine the estimated glucose values 132 by applying weights to the inputs (e.g., feature vectors representing the above-noted data). Those weights may correspond to an underlying model determined using historical data of a user population and/or the person 102. In such implementations, the machine learning model is configured to output data (e.g., feature vectors) indicative of the estimated glucose values 132. The output data (e.g., the feature vectors) can be processed to obtain the estimated glucose values 132. The value prediction engine 304 may use the prediction logic 310 to generate the estimated glucose values 132 during the warmup period 126 and until the warmup period 126 ends.

In one or more implementations, the machine learning model can output a probability that the user's glucose measurements will go above a certain glucose threshold (e.g., above 180 mg/dL) during the transition between sensors to indicate a risk of hyperglycemia and/or a probability that the user's glucose measurements will go below a certain glucose threshold (e.g., below 70 mg/dL) during the transition between sensors to indicate the risk of hypoglycemia.

In this example 300, the data accuracy module 308 determines when a warmup period ends. In one or more implementations, the data accuracy module 308 determines an end of a warmup period based on a predetermined amount of time (e.g., substantially no time needed for warmup, less than an hour, such as 30 minutes, two hours, greater than two hours, etc.) after detection that a sensor is inserted into the person 102. The predetermined amount of time may be set based on a myriad of real-world, controlled tests that indicate the glucose sensors will satisfy a threshold amount of accuracy after the predetermined amount of time, absent some defect. In the context of FIG. 1, for instance, the data accuracy module 308 may determine that the warmup period 126 ends a predetermined amount of time after detecting that the glucose sensor of the wearable glucose monitoring device 104 is inserted into the person 102. Alternatively or additionally, the data accuracy module 308 may determine an end of a warmup period based on a determination that the data of the second data stream 120 describes the glucose of person 102 accurately, e.g., an accuracy of the second data stream 120 satisfies an accuracy threshold.

In one or more implementations, for instance, the prediction logic 310 may determine an accuracy of the second data stream 120's data based on glucose predictions generated by the prediction logic 310 (e.g., using the first data stream 118) and also based on the data of the second data stream 120. By way of example, the data accuracy module 308 may compare the second data stream 120's data to the predictions and determine accuracy of the second data stream 120's data based, in part, on the comparison. For instance, the data accuracy module 308 may identify differences between the predictions and the second data stream 120's data by comparing them. Based on those differences, the data accuracy module 308 can further determine when the data of the second data stream 120 remains within a range of the glucose predictions generated by the prediction logic 310 for some threshold amount of time, with some threshold frequency, and/or for some threshold number of measurements.

Alternatively or additionally, the data accuracy module 308 may compute one or more metrics in relation to the second data stream 120's data and determine accuracy of the second data stream 120 based on the metrics. For instance, the data accuracy module 308 may compute a variability of the second glucose measurements 130(a) of the second data stream 120. In this scenario, when the data accuracy module 308 determines that the computed variability satisfies an accuracy threshold, then the data accuracy module 308 may also determine that the warmup period 126 is ended. In one or more implementations, the data accuracy module 308 may generate measure of accuracy based on one or more of the techniques described above and or using other techniques. It is to be appreciated the data accuracy module 308 may determine accuracy in the data streams produced by and received from a sensor in a variety of ways without departing from the spirit or scope of the described techniques.

By using accuracy of the data of the second data stream 120 to determine the warmup period 126, the warmup period 126 may be shortened in relation to a predetermined amount of time. For example, the warmup period 126 may be shortened from a first predetermined amount of time (e.g., two-hour, 30 minutes, etc.) to an amount of time that is less than the first predetermined amount of time (e.g., less than two-hours, less than 30 minutes, etc., respectively). Using glucose predictions for the warmup period 126 that are based on the first data stream 118, may also shorten the warmup period 126 in relation to a predetermined amount of time. In so doing, the computing device 106 may present the person 102's glucose to them (or another user associated with the person 102) sooner than if a predetermined amount of time is used for the warmup period 126. This enables the person 102, a health care provider, or another user to take action to manage the person 102's glucose levels sooner than if a predetermined amount of time is used for the warmup period 126. By presenting accurate glucose sooner, potentially dangerous events in relation to the person 102's health may be avoided. This also enables people relying on wearable glucose monitoring devices 104 to reduce finger sticks as compared to when predetermined amounts of time are used. This eliminates a potentially painful and annoying activity from their lives and adds to quality of life.

The illustrated example 300 also includes display module 314, which is depicted receiving the estimated glucose values 132 as input. In general, the display module 314 is configured to generate a user interface 316 that displays a glucose presentation 318. In one or more implementations, the user interface 316 is displayed via a display device of the computing device 106, and the user interface 316 may be displayed within an application.

Such an application may correspond to a glucose monitoring platform (e.g., a provider of the wearable glucose monitoring device 104) and run on the computing device 106, such as in the background and/or responsive to selection of an icon via the display device of the computing device 106. Moreover, such an application may correspond to a web application that interacts with a glucose monitoring platform over the network 110 to provide a variety of functionality.

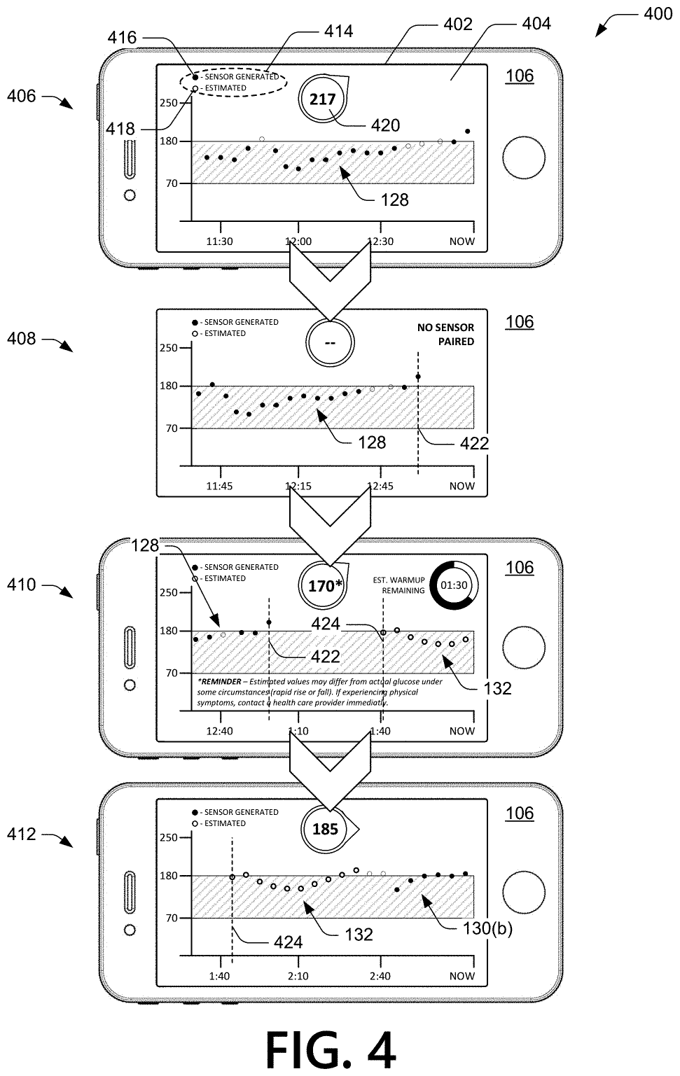

The glucose presentation 318 may be configured in a variety of ways to display one or more of the first glucose measurements 128, the estimated glucose values 132, and/or the second glucose measurements 130(a), 130(b). For example, the glucose presentation 318 may be configured as one or more glucose traces, plotted over time. In one or more implementations, the glucose presentation 318 may also be configured to display the second glucose measurements 130(a) concurrently with the estimated glucose values 132. By displaying both, the glucose presentation 318 visually conveys an accuracy (or inaccuracy) of the actual measurements produced during the warmup period 126 and/or a difference between the actual measurements and the estimated glucose values 132. Alternatively or additionally, the display module 314 may determine not to display glucose measurements or estimated glucose values in a variety of scenarios, such as when a difference between the estimated glucose values 132 and the second glucose measurements 130(a) exceeds a threshold. In the context of displaying a user interface that includes a glucose presentation, consider the following discussion of FIG. 4.

FIG. 4 depicts an example 400 of an implementation of a user interface displaying plots of a user's glucose over time including estimated glucose values for a warmup period of a glucose sensor.

The example 400 depicts an example of the computing device 106. Here, the computing device includes display device 402, which is depicted outputting user interface 404 for display at four chronologically sequenced stages 406-412, including a first stage 406, a second stage 408, a third stage 410, and a fourth stage 412. It is to be appreciated that the user interface 404 may correspond to the user interface 316. Accordingly, the display module 314 may cause output of the user interface 404 for display.

At the first stage 406, the user interface 404 includes a graph that plots the first glucose measurements 128 over time. This may correspond to an example of the glucose presentation 318. Here, the user interface 404 also includes a key 414, which identifies a first symbol 416 and a second symbol 418. In this example 400, the first symbol 416 represents glucose measurements which have been produced by a glucose sensor, and the second symbol 418 represents estimated glucose values, e.g., estimated by the prediction logic 310 based on glucose measurements produced by a glucose sensor. Notably, the graph presented via the user interface 404 at the first stage 406 only displays glucose measurements, which is indicated by the plurality of first symbols 416 plotted on the graph. Further, the graph depicted at the first stage 406 does not display any estimated glucose values, which is indicated by the absence of the second symbol 418 on the graph.

The user interface 404 also includes current glucose element 420. In general, the current glucose element 420 displays a glucose measurement or an estimated glucose value that corresponds to a current time, e.g., a most recent glucose measurement produced or a most recent glucose value estimated. When a user is not wearing a glucose sensor though, the current glucose element 420 may display one or more symbols (e.g., or ‘N/A’) indicating that no measurement or estimated value is available for display in the current glucose element 420. Alternatively, the current glucose element 420 may display an estimated glucose value when a user is not wearing a glucose sensor.

In this example 400, the second stage 408 corresponds to a point in time that is subsequent to an earlier point in time corresponding to the first stage 406. At the second stage 408, the user interface 404 is depicted displaying a plurality of the first glucose measurements 128 that were displayed at the first stage 406. However, the graph does not depict symbols between time 422 and a current time, which is indicated by the text ‘NOW’ along the graph's x-axis. This may indicate that a corresponding user is not wearing a glucose sensor, such as after removing a first sensor and before applying a second, subsequent sensor. In the context of FIG. 1, for example, the user interface as displayed at the second stage 408 may correspond to the person 102 at the second stage 114, e.g., after removing the wearable glucose monitoring device 104(a) but before applying the wearable glucose monitoring device 104(b). At the second stage 408, the current glucose element 420 does not display a value indicative of a corresponding user's glucose. Instead, the current glucose element 420 displays at the second stage 408, which, as noted above, may indicate that the user is not currently wearing a sensor or that glucose measurements are not being received by the computing device 106 from the sensor.

The third stage 410 corresponds to a point in time that is subsequent to the point in time corresponding to the second stage 408. At the third stage 410, the user interface 404 is depicted displaying a plurality of the first glucose measurements 128 that were displayed at the first and second stages 406, 408, but fewer of those measurements than either preceding stage. In this example 400, there are no glucose symbols depicted between time 422 and subsequent time 424. In accordance with the described techniques, this gap between symbols may correspond to a time period between wearing sensors, i.e., after a first sensor is removed and before a second sensor is applied. In addition, the user interface 404 is depicted displaying a plurality of the estimated glucose values 132 at the third stage 410. This may indicate that the corresponding user is wearing a second sensor (e.g., the sensor of the wearable glucose monitoring device 104(b)), but that glucose values are being estimated and displayed rather than displaying measurements produced by and received from the second sensor. This third stage 410 may correspond to a point in time during the warmup period 126 of the second sensor, for example, where the estimated glucose values 132 are displayed in place of the second glucose measurements 130(a), e.g., because the second glucose measurements 130(a) do not satisfy an accuracy threshold.

At the third stage 410, the current glucose element 420 displays a glucose value with an additional information indicator ‘*’. In accordance with the described techniques, the additional information indicator in the current glucose element 420 can serve as a visual “warning” or “alert,” indicating that there is pertinent information relative to a value or symbols displayed in the current glucose element 420. Here, the additional information indicator corresponds to a warning that reminds a user that the estimated glucose values 132 are estimated and may not correspond to the user's actual glucose in some circumstances, such as when the user's actual glucose changes rapidly or due to an event that is not accounted for by the prediction logic 310. The current glucose element 420 may include additional information indicators in connection with displaying different, pertinent information without departing from the spirit or scope of the described techniques.

At the third stage 410, the user interface 404 also displays remaining time indicator 426. The remaining time indicator 426 may indicate an actual amount of time remaining of the warmup period 126, such as when the warmup period 126 is configured to last a predetermined amount of time, e.g., two hours, 30 minutes, etc. Alternatively or additionally, the remaining time indicator 426 may indicate an estimated amount of time remaining of the warmup period 126, such as when the data accuracy module 308 determines an end of the warmup period 126 based on an accuracy of glucose measurements produced by and received from a respective sensor satisfying an accuracy threshold. The remaining time indicator 426 may include numerical values indicating a remaining amount of time and/or non-numerical visual elements indicating a remaining amount of time. The remaining time indicator 426 may indicate an actual or estimated amount of remaining time in a variety of ways without departing from the spirit or scope of the described techniques, including visually, audibly, and/or haptically.

The fourth stage 412 corresponds to a point in time that is subsequent to the point in time corresponding to the third stage 410. At the fourth stage 412, the user interface 404 is depicted displaying a plurality of the estimated glucose values 132 that were displayed at the third stage 410 along with additional values of the estimated glucose values 132. To the left of the time 424, no symbols are displayed in the user interface 404's graph at the fourth stage 412. The leftward shift of this gap in symbols and of the estimated glucose values 132 across stages 408-412 indicates the passage of time. At the fourth stage 412, the user interface 404 also displays the second glucose measurements 130(b), which, in the context of FIG. 1, correspond to a period of time after warmup period 126. In accordance with the described techniques, the display module 314 may display the second glucose measurements 130(b) after a glucose sensor has been deployed for the warmup period 126, which lasts until a predetermined amount of time lapses or responsive to a determination by the data accuracy module 308 that an accuracy of the measurements satisfies an accuracy threshold.

FIG. 5 depicts an example 500 of a scenario in which a bridging system generates estimated glucose values during a transition period between sensors.

The illustrated example 500 includes the person 102 depicted at the first, second, and third stages 112-116, along with the computing device 106, and the bridging system 108. The illustrated example 500 also depicts the first glucose measurements 128 of the first data stream 118 and the second glucose measurements 130(a), 130(b) of the second data stream 120. In this example 500, the bridging system 108 is also depicted outputting the estimated glucose values 132 for the warmup period 126, in similar manner as in FIG. 1.

In contrast to FIG. 1 though, the bridging system 108 is depicted outputting a second set of estimated glucose values 502 in this example 500. In this example 500, the second set of estimated glucose values 502 correspond to the second stage 114—a time period during which the person 102 is not wearing a glucose sensor. This may correspond to a time that the user transitions between sensors, e.g., transitions from a first sensor (e.g., of the wearable glucose monitoring device 104(a)) to a second senor (e.g., of the wearable glucose monitoring device 104(b)). Said another way, the bridging system 108 may generate estimated glucose values for a time period where the beginning corresponds to removal the wearable glucose monitoring device 104(a) from the person 102 and where the end corresponds to deployment of the wearable glucose monitoring device 104(b). In contrast to the estimated glucose values 132, the second set of estimated glucose values 502 may be generated based on only the first glucose measurements 128. This is because no sensor may be deployed in the person 102 for the time period corresponding to the second set of estimated glucose values 502 is determined. For instance, the glucose sensor of the wearable glucose monitoring device 104(b) may not yet be deployed when the bridging system 108 determines and causes output of the second set of estimated glucose values 502.

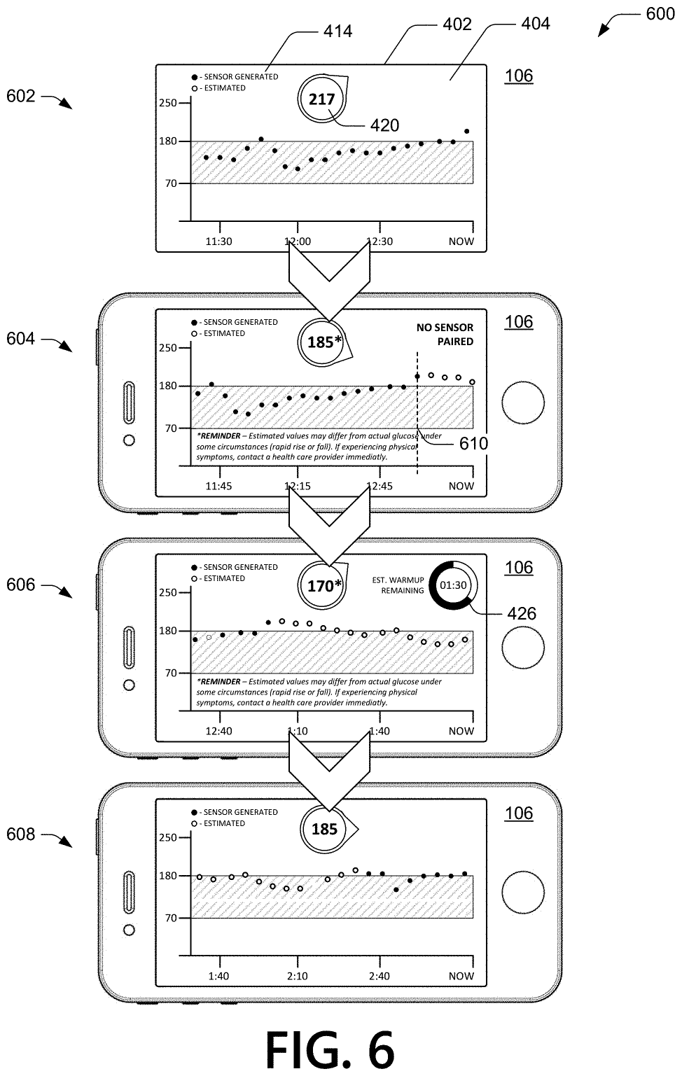

In accordance with the described techniques, the second set of estimated glucose values 502 may correspond to predictions of glucose generated by the prediction logic 310 based on the first glucose measurements 128 of the first data stream 118. In contrast to the estimated glucose values 132, the prediction logic 310 may determine the second set of estimated glucose values 502 without using the second glucose measurements 130(a). Indeed, the prediction logic 310 may determine the second set of estimated glucose values 502 before the second glucose measurements 130(a) are even produced, e.g., because the wearable glucose monitoring device 104(a) may not be deployed yet when the second set of estimated glucose values 502 are determined. In this scenario, the prediction logic 310 may generate the glucose predictions used as the second set of estimated glucose values 502 using one or more machine learning models, such as one or more general models configured to generate predictions for users of a user population and/or one or more user-specific models configured to generate predictions for a specific user. In the context of displaying a set of estimated glucose values during a transition between sensors, consider the following discussion of FIG. 6.

FIG. 6 depicts an example 600 of an implementation of a user interface displaying plots of a user's glucose over time including during a transition period between glucose sensors.

The illustrated example 600 depicts another example of the computing device having the display device 402. In this example 600, the display device 402 is depicted outputting the user interface 404 at four chronologically sequenced stages 602-608, including a first stage 602, a second stage 604, a third stage 606, and a fourth stage 608. The user interface 404 may correspond to the user interface 316 and the display module 314 may cause output of the user interface 404. In contrast to the example depicted in FIG. 4, however, the stages 602-608 may plot glucose for the corresponding user when the use transitions between glucose sensors, including when the user is not wearing a glucose sensor.

In this example 600, the first stage 602 is similar to the first stage 406 depicted in FIG. 4. At the first stage 602, for example, the user interface 404 includes a graph that plots the first glucose measurements 128 over time. Presentation of the graph with plotted glucose may correspond to another example of the glucose presentation 318. Notably, the graph presented via the user interface 404 at the first stage 602 only displays glucose measurements and does not display any estimated glucose values.

The second stage 604 in this example 600 corresponds to a point in time that is subsequent to an earlier point in time corresponding to the first stage 602. In a similar manner as the second stage 408 of FIG. 4, the user interface 404 is depicted displaying a plurality of the first glucose measurements 128 at the second stage 604 that were previously displayed at the first stage 602. Here, the user interface 404 is depicted displaying a plurality of the first glucose measurements 128 up to time 610. The time 610 may correspond to the termination event 124, when the person 102 removes the wearable glucose monitoring device 104(a) and its respective glucose sensor, ceasing production and transmission of glucose measurements.

In contrast to the second stage 408 of FIG. 4 though, the user interface 404 is depicted displaying the second set of estimated glucose values 502 between the time 610 and a current time, which is indicated by the text ‘NOW’ along the graph's x-axis. This contrasts with the second stage 408 of FIG. 4 because the second stage 408 of FIG. 4 depicts no symbols during this same time period. The stages 602-608 thus depict a different scenario from the stages 406-412 of FIG. 4, namely, the stages 602-608 depict a scenario where the bridging system 108 is configured to predict glucose values for time periods while a user does not wear a sensor and to cause display of those values.

Since the user interface 404 at the second stage 604 displays such predicted values, the current glucose element 420 displays a glucose value with an additional information indicator at the second stage 604. In this scenario, the additional information indicator can serve as a visual “warning” or “alert,” indicating that there is pertinent information relative to a value or symbols displayed in the current glucose element 420. Here, the additional information indicator corresponds to a warning that reminds a user that the second set of estimated glucose values 502 are estimated and may not correspond to the user's actual glucose in some circumstances, such as when the user's actual glucose changes rapidly or due to an event that is not accounted for by the prediction logic 310.

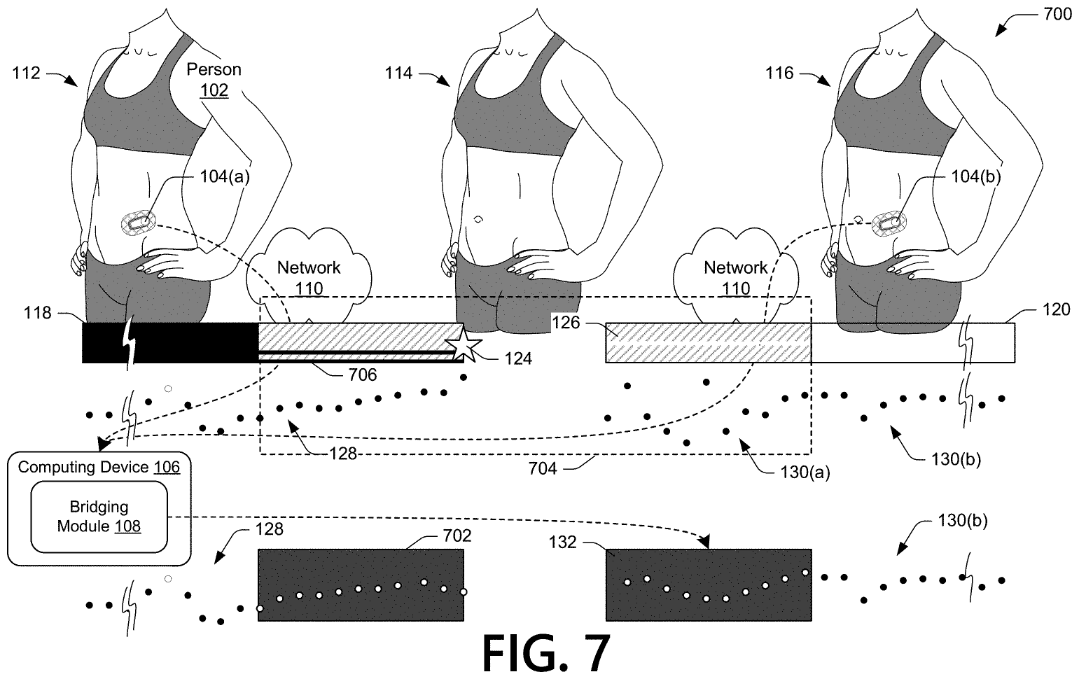

The third and fourth stages 606, 608 are similar to the third and fourth stages 410, 412 of FIG. 4, with the exception that the third and fourth stages 606, 608 display the second set of estimated glucose values 502—rather than display no measurements or values while a user transitions between sensors. This may enable a user or health care provider to continue making treatment decisions even for periods of time while a user does not wear a glucose sensor, e.g., while transitioning between sensors. In the context of retrospectively updating at least one glucose sensor value of data stream of glucose measurements, consider the following discussion of FIG. 7.

FIG. 7 depicts an example 700 of a scenario in which a bridging system generates estimated glucose values retrospectively to replace glucose measurements at an end of a first sensor's data stream.

The illustrated example 700 includes from the person 102 depicted at the first, second, and third stages 112-116, along with the computing device 106, and the bridging system 108. The illustrated example 700 also depicts the first glucose measurements 128 of the first data stream 118 and the second glucose measurements 130(a), 130(b) of the second data stream 120. In this example 700, the bridging system 108 is also depicted outputting the estimated glucose values 132 for the warmup period 126, in similar manner as in FIG. 1.

In contrast to FIG. 1 though, the bridging system 108 is depicted outputting retrospective glucose values 702 in this example 700. The transition period 704 in this example 700 also spans a different amount of time than the transition period 122 of FIG. 1. The transition period 122 of FIG. 1 begins at the termination event 124 and lasts until an end of the warmup period 126. By contrast, the transition period 704, as illustrated, begins at a beginning of the cool-down period 706 and lasts until an end of the warmup period 126. This cool-down period 706 may correspond to a predetermined amount of time at an end of a sensor's life. Alternatively, the data accuracy module 308 (or a different module that is configured to calculate confidence in the accuracy of glucose measurements) may determine a beginning of the cool-down period 706 based on an accuracy of the first glucose measurements 128, such that the cool-down period 706 is determined to begin when the first glucose measurements 128 fail to satisfy a threshold accuracy, e.g., based on unexpected variability in the sensor signal. Regardless of whether the cool-down period 706 corresponds to a predetermined amount of time or is determined based on accuracy of the measurements, the bridging system 108 may be configured to retrospectively update the first glucose measurements 128 that correspond to the cool-down period 706.

In one or more implementations, the bridging system 108 may determine the retrospective glucose values 702 based on the first glucose measurements 128 produced during the cool-down period 706 and also based on one or more of the second glucose measurements 130(a), 130(b). In one or more scenarios, the data accuracy module 308 may not determine that the first glucose measurements 128 fail to meet the threshold accuracy until the computing device 106 receives the second glucose measurements 130(a), 130(b). Indeed, the bridging system 108 may determine the retrospective glucose values 702 at a time when the glucose sensor of the wearable glucose monitoring device 104(b) produces glucose values and communicates them to the computing device 106. In one or more implementations, the prediction logic 310 may thus be configured to retrospectively generate glucose values, e.g., given as input measurements preceding the prediction (e.g., one or more of the first glucose measurements 128 prior to the cool-down period 706), measurements that correspond to a same time as the prediction (e.g., the first glucose measurements 128 corresponding to the cool-down period 706), and measurements subsequent to the prediction (e.g., one or more of the second glucose measurements 130(a), 130(b)). The prediction logic 310 may use one or more machine learning models and/or a weighted average to generate the retrospective glucose values 702 for the cool-down period 706.

Having discussed exemplary details of the techniques for data-stream bridging for sensor transitions, consider now some examples of procedures to illustrate additional aspects of the techniques.

### Example Procedures

This section describes examples of procedures for data-stream bridging for sensor transitions. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations the procedures are performed by a bridging system, such as the bridging system 108 that makes use of the stream handler 302 and the value prediction engine 304.

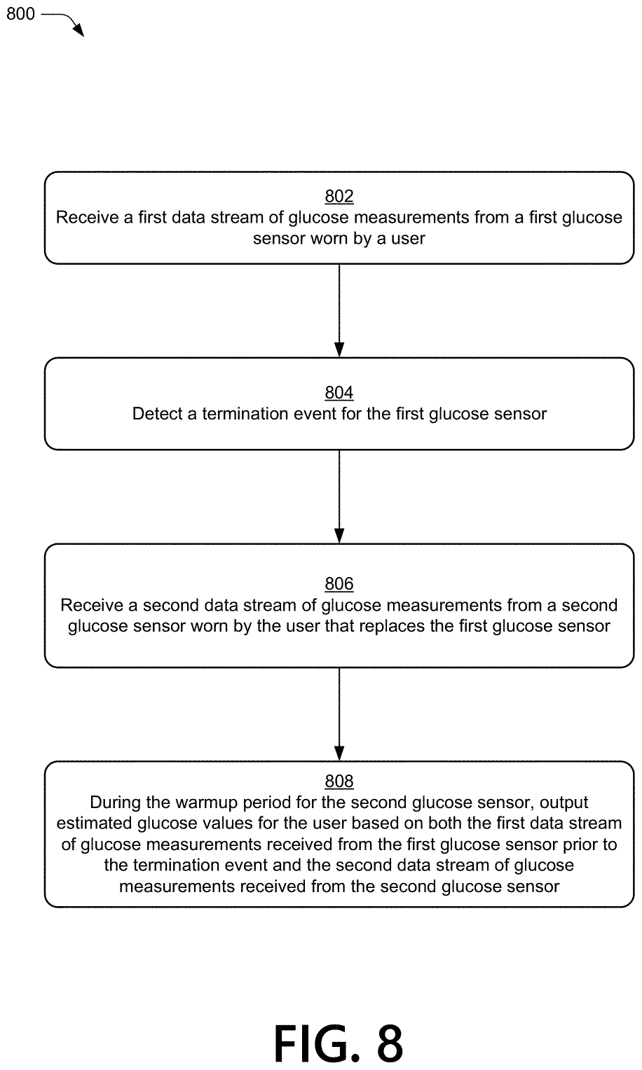

FIG. 8 depicts a procedure 800 in an example implementation in which estimated glucose values are output based on both a first stream of glucose measurements received from a first glucose sensor prior to a termination event and a second data stream of glucose measurements received from a second glucose sensor that replaces the first glucose sensor or is intended to replace the first glucose sensor at some point in time.

A first data stream of glucose measurements is received from a first glucose sensor worn by a user (block 802). By way of example, the bridging system 108 receives a first data stream 118 of first glucose measurements 128 from a first glucose sensor of wearable glucose monitoring device 104(a) worn by a person 102.

A termination event for the first glucose sensor is detected (block 804). By way of example, termination detection engine 306 detects a termination event 124 for the first glucose sensor. As mentioned above, the termination event 124 corresponds to a time when production and/or communication of the first glucose measurements 128 by the wearable glucose monitoring device 104(a) as part of the first data stream 118 ceases or is due to cease. In different implementations, the termination detection engine 306 may be configured to detect different types of termination events.

By way of example, glucose sensors may be configured for use over a limited, predetermined amount of time, e.g., 7 days, 10 days, or 30 days, to name just a few. In such implementations, each glucose sensor may be associated with a timer, e.g., that begins when the glucose sensor is inserted into a user or is otherwise deployed to measure the user's glucose. The termination detection engine 306 may maintain the timer and/or detect when the timer expires. For instance, when the timer counts down (decays), the termination detection engine 306 may detect when the timer reaches zero or falls to some other threshold value indicating an end of life of the respective sensor. In contrast, when the timer counts up (increases), the termination detection engine 306 may detect when the timer reaches a threshold value (e.g., 10 days) indicating an end of life of the respective sensor.

Alternatively or additionally, the termination detection engine 306 may detect termination events that correspond to changes in conditions that are different from identifying an expiring timer value for a “lifespan” of a glucose sensor. By way of example, the termination detection engine 306 may detect termination events based on loss of connectivity (e.g., communicative or physical) with a glucose sensor. For instance, the termination detection engine 306 may detect that a termination event has occurred when a glucose measurement is not received after a threshold amount of time from a glucose sensor, when a signal (e.g., a “heartbeat”) is not received from the glucose sensor), when a signal is received indicating an end of connectivity with the glucose sensor, when an end-of-life chemical reaction is detected for a glucose sensor, or when a chemical reaction is detected for a new sensor, to name just a few. As noted above, glucose sensors of the wearable glucose monitoring devices 104 may be inserted subcutaneously into the person 102's skin. In one or more implementations, the termination detection engine 306 may be configured to detect termination events when glucose sensors are removed from the skin of the user, e.g., withdrawn from the person 102's skin.

A second data stream of glucose measurements is received from a second glucose sensor worn by the user that replaces (e.g., immediately or eventually) the first glucose sensor (block 806). By way of example, the bridging system 108 receives a second data stream 120 of glucose measurements 130(a) from a second glucose sensor of wearable glucose monitoring device 104(b) worn by a person 102. The second data stream 120 and the first data stream 118 may be received subsequently to one another and/or simultaneously with one another.

During a warmup period for the second glucose sensor, estimated glucose values for the user are output based on both the first data stream of glucose measurements received from the first glucose sensor (e.g., prior to the termination event) and the second data stream of glucose measurements received from the second glucose sensor (block 808). By way of example, during a warmup period 126 for the second glucose sensor of wearable glucose monitoring device 104 (b), value prediction engine 304 outputs estimated glucose values 132 for person 102 based on both the first data stream 118 of first glucose measurements 128 received from the first glucose sensor prior to the termination event 124 and the second data stream 120 of glucose measurements 130(a) received from the second glucose sensor.

In one or more implementations, the prediction logic 310 may determine the estimated glucose values 132 using one or more weighting techniques, such as weighted averages. For instance, prediction logic 310 may associate relatively smaller weights with the second glucose measurements 130(a) that correspond to the warmup period 126 toward a beginning of the warmup period 126, and the prediction logic 310 may associate relatively larger weights with the second glucose measurements 130(a) that correspond to the warmup period 126 closer to an end of the warmup period 126. In one or more implementations, the prediction logic 310 may associate progressively larger weights with the second glucose measurements 130(a) that correspond to the warmup period 126 over time.

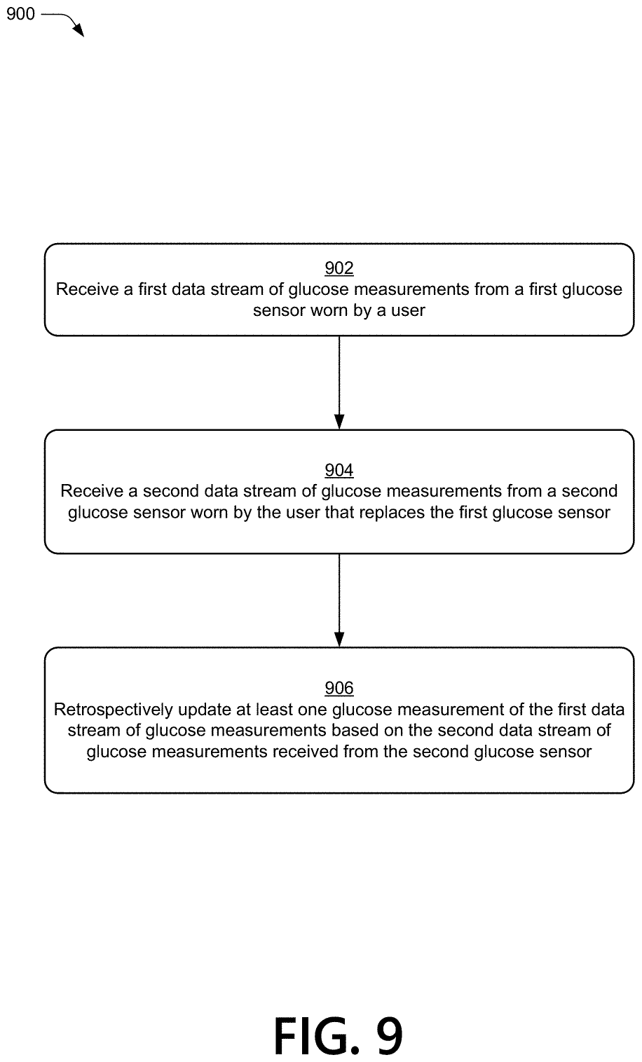

FIG. 9 depicts a procedure 900 in an example implementation in which glucose measurements associated with a first glucose sensor are retrospectively updated based on glucose measurements received from a second glucose sensor that replaces the first glucose sensor.

A first data stream of glucose measurements is received from a first glucose sensor worn by a user (block 902). By way of example, the bridging system 108 receives a first data stream 118 of first glucose measurements 128 from a first glucose sensor of wearable glucose monitoring device 104(a) worn by a person 102.

A second data stream of glucose measurements is received from a second glucose sensor worn by the user that replaces the first glucose sensor (block 904). By way of example, the bridging system 108 receives a second data stream 120 of glucose measurements 130(a) from a second glucose sensor of wearable glucose monitoring device 104(b) worn by person 102 that replaces the first glucose sensor.

At least one glucose measurement of the first data stream of glucose measurements is retrospectively updated based on the second data stream of glucose measurements received from the second glucose sensor (block 906). By way of example, the transition period between sensors may include a cool-down period 706 that corresponds to a predetermined amount of time at an end of a sensor's life. Alternatively, the data accuracy module 308 may determine a beginning of the cool-down period 706 based on an accuracy of the first glucose measurements 128, such that the cool-down period 706 is determined to begin when the first glucose measurements 128 fail to satisfy a threshold accuracy. Regardless of whether the cool-down period 706 corresponds to a predetermined amount of time or is determined based on accuracy of the measurements, the bridging system 108 may be configured to retrospectively update at least one of the first glucose measurements 128 that correspond to the cool-down period 706.

The bridging system 108 may determine the retrospective glucose values 702 based on the first glucose measurements 128 produced during the cool-down period 706 and also based on one or more of the second glucose measurements 130(a), 130(b). In one or more scenarios, the data accuracy module 308 may not determine that the first glucose measurements 128 fail to meet the threshold accuracy until the computing device 106 receives the second glucose measurements 130(a), 130(b). Indeed, the bridging system 108 may determine the retrospective glucose values 702 at a time when the glucose sensor of the wearable glucose monitoring device 104(b) produces glucose values and communicates them to the computing device 106.

In one or more implementations, the prediction logic 310 may thus be configured to retrospectively generate glucose values, e.g., given as input measurements preceding the prediction (e.g., one or more of the first glucose measurements 128 prior to the cool-down period 706), measurements that correspond to a same time as the prediction (e.g., the first glucose measurements 128 corresponding to the cool-down period 706), and measurements subsequent to the prediction (e.g., one or more of the second glucose measurements 130(a), 130(b)). The prediction logic 310 may use one or more machine learning models and/or a weighted average to generate the retrospective glucose values 702 for the cool-down period 706.

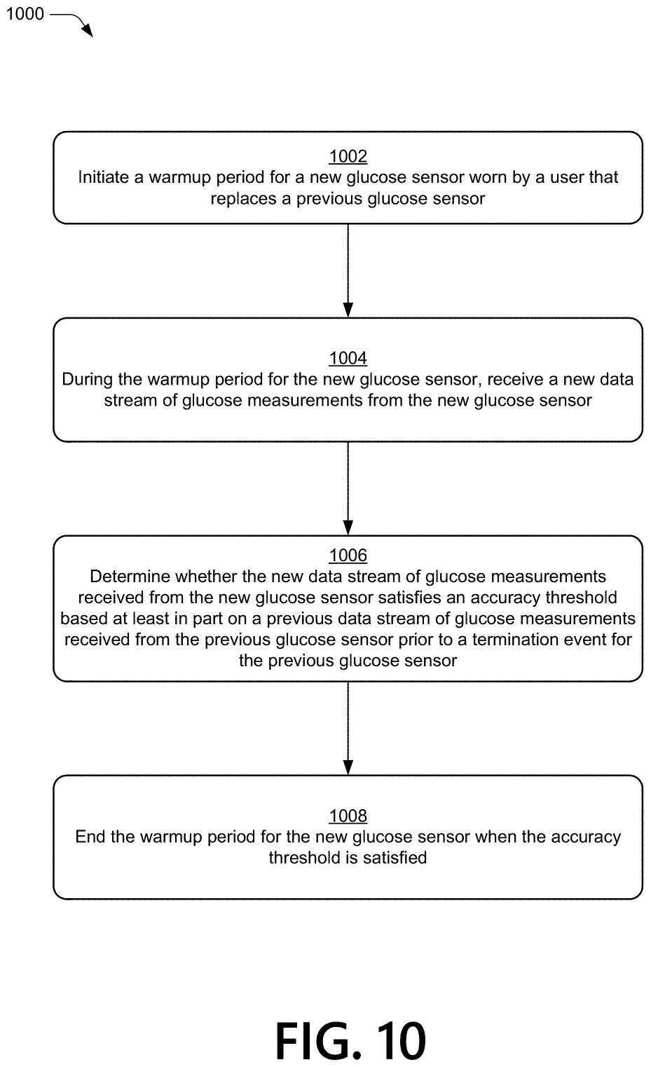

FIG. 10 depicts a procedure 1000 in an example of an implementation in which a warmup period for a new glucose sensor is ended when glucose measurements received from the new glucose sensor satisfy an accuracy threshold.

A warmup period for a new glucose sensor worn by the user that replaces a previous glucose sensor is initiated (block 1002). During the warmup period for the new glucose sensor, a new data stream of glucose measurements is received from the new glucose sensor (block 1004), and it is determined whether the new data stream of glucose measurements received from the new glucose sensor satisfy an accuracy threshold based at least in part on a previous data stream of glucose measurements received from the previous glucose sensor prior to a termination event for the previous glucose sensor (block 1006). By way of example, the prediction logic 310 may determine an accuracy of the second data stream 120's data based on glucose predictions generated by the prediction logic 310 (e.g., using the first data stream 118) and also based on the data of the second data stream 120. The data accuracy module 308 may compare the second data stream 120's data to the predictions and determine accuracy of the second data stream 120's data based, in part, on the comparison.

For instance, the data accuracy module 308 may identify differences between the predictions and the second data stream 120's data by comparing them. Based on those differences, the data accuracy module 308 can further determine when the data of the second data stream 120 remains within a range of the glucose predictions generated by the prediction logic 310 for some threshold amount of time, with some threshold frequency, and/or for some threshold number of measurements. Alternatively or additionally, the data accuracy module 308 may compute one or more metrics in relation to the second data stream 120's data and determine accuracy of the second data stream 120 based on the metrics. For instance, the data accuracy module 308 may compute a variability of the second glucose measurements 130(a) of the second data stream 120.

The warmup period for the new glucose sensor is ended when the accuracy threshold is satisfied (block 1008). By way of example, when the data accuracy module 308 determines that the computed variability satisfies an accuracy threshold, then the data accuracy module 308 may also determine that the warmup period 126 is ended. Notably, by using accuracy of the data of the second data stream 120 to determine the warmup period 126, the warmup period 126 may be shortened in relation to a predetermined amount of time. The above-described systems and procedures are usable in various combinations to implement different methods without departing from the spirit or scope of the described techniques. Indeed, at least one method may include different aspects from the described procedures and/or the described systems in accordance with the described techniques.

Having described example procedures in accordance with one or more implementations, consider now an example of a system and device that can be utilized to implement the various techniques described herein.

### Example System and Device

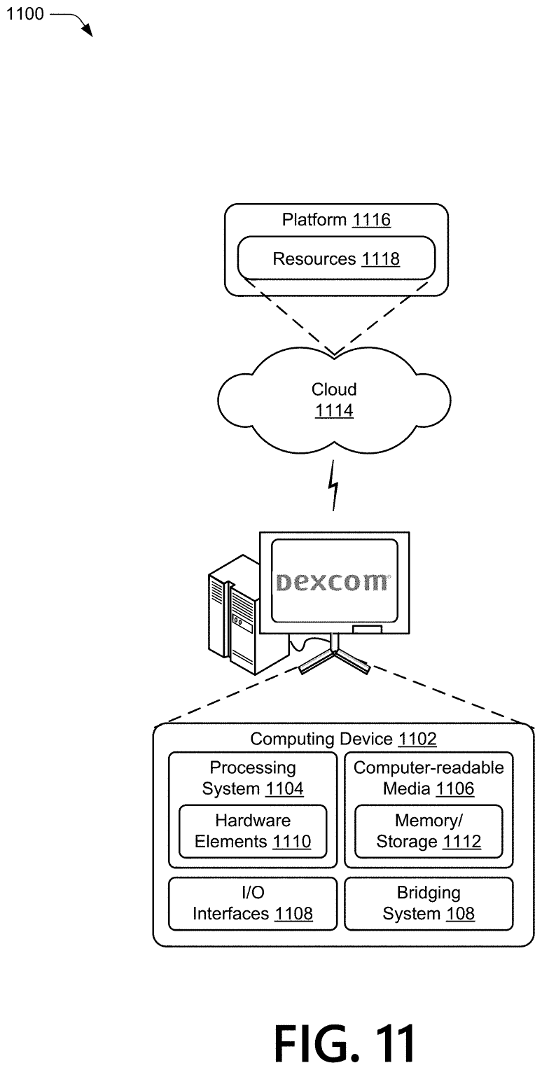

FIG. 11 illustrates an example of a system generally at 1100 that includes an example of a computing device 1102 that is representative of one or more computing systems and/or devices that may implement the various techniques described herein. This is illustrated through inclusion of the bridging system 108. The computing device 1102 may be, for example, a server of a service provider, a device associated with a client (e.g., a client device), an on-chip system, and/or any other suitable computing device or computing system.

The example computing device 1102 as illustrated includes a processing system 1104, one or more computer-readable media 1106, and one or more I/O interfaces 1108 that are communicatively coupled, one to another. Although not shown, the computing device 1102 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1104 is representative of functionality to perform one or more operations using hardware. Accordingly, the processing system 1104 is illustrated as including hardware elements 1110 that may be configured as processors, functional blocks, and so forth. This may include implementation in hardware as an application specific integrated circuit or other logic device formed using one or more semiconductors. The hardware elements 1110 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 1106 is illustrated as including memory/storage 1112. The memory/storage 1112 represents memory/storage capacity associated with one or more computer-readable media. The memory/storage 1112 may include volatile media (such as random access memory (RAM)) and/or nonvolatile media (such as read only memory (ROM), Flash memory, optical disks, magnetic disks, and so forth). The memory/storage 1112 may include fixed media (e.g., RAM, ROM, a fixed hard drive, and so on) as well as removable media (e.g., Flash memory, a removable hard drive, an optical disc, and so forth). The computer-readable media 1106 may be configured in a variety of other ways as further described below.

Input/output interface(s) 1108 are representative of functionality to allow a user to enter commands and information to computing device 1102, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1102 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 1102. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1102, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1110 and computer-readable media 1106 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1110. The computing device 1102 may be configured to implement particular instructions and/or functions corresponding to the software and/or hardware modules. Accordingly, implementation of a module that is executable by the computing device 1102 as software may be achieved at least partially in hardware, e.g., through use of computer-readable storage media and/or hardware elements 1110 of the processing system 1104. The instructions and/or functions may be executable/operable by one or more articles of manufacture (for example, one or more computing devices 1102 and/or processing systems 1104) to implement techniques, modules, and examples described herein.

The techniques described herein may be supported by various configurations of the computing device 1102 and are not limited to the specific examples of the techniques described herein. This functionality may also be implemented all or in part through use of a distributed system, such as over a “cloud” 1114 via a platform 1116 as described below.

The cloud 1114 includes and/or is representative of a platform 1116 for resources 1118. The platform 1116 abstracts underlying functionality of hardware (e.g., servers) and software resources of the cloud 1114. The resources 1118 may include applications and/or data that can be utilized while computer processing is executed on servers that are remote from the computing device 1102. Resources 1118 can also include services provided over the Internet and/or through a subscriber network, such as a cellular or Wi-Fi network.

The platform 1116 may abstract resources and functions to connect the computing device 1102 with other computing devices. The platform 1116 may also serve to abstract scaling of resources to provide a corresponding level of scale to encountered demand for the resources 1118 that are implemented via the platform 1116. Accordingly, in an interconnected device embodiment, implementation of functionality described herein may be distributed throughout the system 1100. For example, the functionality may be implemented in part on the computing device 1102 as well as via the platform 1116 that abstracts the functionality of the cloud 1114.

### Conclusion

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A method comprising:
receiving a first data stream of glucose measurements from a first glucose sensor worn by a user;
detecting a termination event for the first glucose sensor;
receiving a second data stream of glucose measurements from a second glucose sensor worn by the user that replaces the first glucose sensor; and
during a warmup period for the second glucose sensor, outputting estimated glucose values for the user based on both the first data stream of glucose measurements received from the first glucose sensor prior to the termination event and the second data stream of glucose measurements received from the second glucose sensor.

2. The method of claim 1, wherein the outputting further comprises:
predicting glucose values for the user during the warmup period based on the first data stream of glucose measurements received from the first glucose sensor prior to the termination event for the first glucose sensor; and
outputting the estimated glucose values based on both the predicted glucose values and the second data stream of glucose measurements received from the second glucose sensor.

3. The method of claim 2, wherein the predicting is further based on at least one of food logging data or activity data.

4. The method of claim 2, wherein the outputting further comprises determining a weighted average between the predicted glucose values and the second data stream of glucose measurements received from the second glucose sensor.

5. The method of claim 2, further comprising, after the warmup period is ended, outputting estimated glucose values based on the second data stream of glucose measurements received in real-time from the second glucose sensor worn by the user.

6. The method of claim 1, wherein the warmup period for the second glucose sensor comprises a predetermined amount of time.

7. The method of claim 1, further comprising:
determining an accuracy of the glucose measurements of the second data stream; and
ending the warmup period for the second glucose sensor when the accuracy of the glucose measurements of the second data stream satisfies an accuracy threshold.

8. The method of claim 7, wherein the accuracy of the glucose measurements of the second data stream is based on a comparison of the glucose measurements of the second data stream to predicted glucose values determined based on the first data stream of glucose measurements received prior to the termination event.

9. The method of claim 1, further comprising:
predicting glucose values for the user based on the first data stream of glucose measurements received from the first glucose sensor prior to the termination event; and
outputting the predicted glucose values for a time period that is after the termination event and before the second glucose sensor is worn by the user.

10. The method of claim 1, wherein the detecting the termination event for the first glucose sensor comprises detecting the termination event when a timer associated with the first glucose sensor expires.

11. The method of claim 1, wherein the detecting the termination event for the first glucose sensor comprises detecting the termination event based on a loss of connectivity with the first glucose sensor or based on user interaction with a user interface.

12. The method of claim 1, wherein the first glucose sensor is subcutaneously inserted into skin of the user, and wherein the termination event is detected in response to the first glucose sensor being removed from the skin of the user.

13. The method of claim 1, wherein the outputting comprises causing display of the estimated glucose values in a user interface of a computing device that is communicatively coupled to the second glucose sensor.

14. The method of claim 1, wherein the first glucose sensor and the second glucose sensor are disposable continuous glucose monitoring (CGM) sensors.

15. A method comprising:
receiving a first data stream of glucose measurements from a first glucose sensor worn by a user;
receiving a second data stream of glucose measurements from a second glucose sensor worn by the user that replaces the first glucose sensor; and
retrospectively updating at least one glucose measurement of the first data stream of glucose measurements based on the second data stream of glucose measurements received from the second glucose sensor.

16. A method comprising:
initiating a warmup period for a new glucose sensor worn by a user that replaces a previous glucose sensor;
during the warmup period for the new glucose sensor, receiving a new data stream of glucose measurements from the new glucose sensor;
determining whether the new data stream of glucose measurements received from the new glucose sensor satisfies an accuracy threshold based at least in part on a previous data stream of glucose measurements received from the previous glucose sensor prior to a termination event for the previous glucose sensor; and
ending the warmup period for the new glucose sensor when the accuracy threshold is satisfied.

17. A method comprising:
outputting estimated glucose values for a user based on a first data stream of glucose measurements received from a first glucose sensor worn by a user;
during a warmup period for a second glucose sensor that replaces the first glucose sensor, outputting estimated glucose values for the user based on the first data stream of glucose measurements received from the first glucose sensor and a second data stream of glucose measurements received from the second glucose sensor; and
after the warmup period for the second glucose sensor ends, outputting estimated glucose values for the user based on the second data stream of glucose measurements received from the second glucose sensor.

18. The method of claim 17, further comprising after a termination event associated with the first glucose sensor and before the warmup period for the second glucose sensor begins, outputting predicted glucose values for the user based on the first data stream of glucose measurements received from the first glucose sensor prior to the termination event.

19. The method of claim 17, further comprising after a termination event associated with the first glucose sensor and before the warmup period for the second glucose sensor begins, preventing the output of predicted glucose values for the user.

20. The method of claim 17, wherein the first glucose sensor and the second glucose sensor are disposable continuous glucose monitoring (CGM) sensors.

21. A system comprising:
one or more processors; and
memory storing computer-readable instructions that are executable by the one or more processors to perform operations including:
receiving a first data stream of glucose measurements from a first glucose sensor worn by a user;
detecting a termination event for the first glucose sensor;
receiving a second data stream of glucose measurements from a second glucose sensor worn by the user that replaces the first glucose sensor; and
during a warmup period for the second glucose sensor, outputting estimated glucose values for the user based on both the first data stream of glucose measurements received from the first glucose sensor prior to the termination event and the second data stream of glucose measurements received from the second glucose sensor.

22. A system comprising:
one or more processors; and
memory storing computer-readable instructions that are executable by the one or more processors to perform operations including:
receiving a first data stream of glucose measurements from a first glucose sensor worn by a user;
receiving a second data stream of glucose measurements from a second glucose sensor worn by the user that replaces the first glucose sensor; and
retrospectively updating at least one glucose measurement of the first data stream of glucose measurements based on the second data stream of glucose measurements received from the second glucose sensor.

23. A system comprising:
one or more processors; and
memory storing computer-readable instructions that are executable by the one or more processors to perform operations including:
initiating a warmup period for a new glucose sensor worn by a user that replaces a previous glucose sensor;
during the warmup period for the new glucose sensor, receiving a new data stream of glucose measurements from the new glucose sensor;
determining whether the new data stream of glucose measurements received from the new glucose sensor satisfies an accuracy threshold based at least in part on a previous data stream of glucose measurements received from the previous glucose sensor prior to a termination event for the previous glucose sensor; and
ending the warmup period for the new glucose sensor when the accuracy threshold is satisfied.

24. A system comprising:
one or more processors; and
memory storing computer-readable instructions that are executable by the one or more processors to perform operations including:
outputting estimated glucose values for a user based on a first data stream of glucose measurements received from a first glucose sensor worn by a user;
during a warmup period for a second glucose sensor that replaces the first glucose sensor, outputting estimated glucose values for the user based on the first data stream of glucose measurements received from the first glucose sensor and a second data stream of glucose measurements received from the second glucose sensor; and
after the warmup period for the second glucose sensor ends, outputting estimated glucose values for the user based on the second data stream of glucose measurements received from the second glucose sensor.

25. A non-transitory computer-readable medium storing instructions that are executable by one or more processors to perform operations comprising:
receiving a first data stream of glucose measurements from a first glucose sensor worn by a user;
detecting a termination event for the first glucose sensor;
receiving a second data stream of glucose measurements from a second glucose sensor worn by the user that replaces the first glucose sensor; and
during a warmup period for the second glucose sensor, outputting estimated glucose values for the user based on both the first data stream of glucose measurements received from the first glucose sensor prior to the termination event and the second data stream of glucose measurements received from the second glucose sensor.

26. A non-transitory computer-readable medium storing instructions that are executable by one or more processors to perform operations comprising:
receiving a first data stream of glucose measurements from a first glucose sensor worn by a user;
receiving a second data stream of glucose measurements from a second glucose sensor worn by the user that replaces the first glucose sensor; and
retrospectively updating at least one glucose measurement of the first data stream of glucose measurements based on the second data stream of glucose measurements received from the second glucose sensor.

27. A non-transitory computer-readable medium storing instructions that are executable by one or more processors to perform operations comprising:
initiating a warmup period for a new glucose sensor worn by a user that replaces a previous glucose sensor;
during the warmup period for the new glucose sensor, receiving a new data stream of glucose measurements from the new glucose sensor;
determining whether the new data stream of glucose measurements received from the new glucose sensor satisfies an accuracy threshold based at least in part on a previous data stream of glucose measurements received from the previous glucose sensor prior to a termination event for the previous glucose sensor; and
ending the warmup period for the new glucose sensor when the accuracy threshold is satisfied.

28. A non-transitory computer-readable medium storing instructions that are executable by one or more processors to perform operations comprising:
outputting estimated glucose values for a user based on a first data stream of glucose measurements received from a first glucose sensor worn by a user;
during a warmup period for a second glucose sensor that replaces the first glucose sensor, outputting estimated glucose values for the user based on the first data stream of glucose measurements received from the first glucose sensor and a second data stream of glucose measurements received from the second glucose sensor; and
after the warmup period for the second glucose sensor ends, outputting estimated glucose values for the user based on the second data stream of glucose measurements received from the second glucose sensor.
